(19) United States
(12) Patent Application Publication
(10) Pub. No.: US 2021/0205397 A1
(43) Pub. Date: Jul. 8, 2021

(54) METHODS FOR SUPPORTING WEIGHT LOSS AND WEIGHT LOSS MAINTENANCE BY ADMINISTRATION OF AQUATIC PLANTS AND/OR AUTOLOGOUS MICROBIOME SAMPLES

(71) Applicants: Hinoman Ltd., Rishon LeZion (IL); B.G. Negev Technologies & Applications Ltd., at Ben-Gurion University, Beer-Sheva (IL)

(72) Inventors: Iris SHAI, Gedera (IL); Ron SALPETER, Givatayim (IL)

(73) Assignees: Hinoman Ltd., Rishon LeZion (IL); B.G. Negev Technologies & Applications Ltd., at Ben-Gurion University, Beer-Sheva (IL)

(21) Appl. No.: 17/273,382

(22) PCT Filed: Sep. 4, 2019
(86) PCT No.: PCT/IL2019/050987

§ 371 Date: Mar. 4, 2021

(51) Int. Cl. A61K 36/888; A61K 35/742; A61K 35/741; A61K 35/745; A61P 3/04; A61K 36/02; A23L 33/105; A23L 17/60; A23L 33/135; A23L 33/00; A23L 2/52

(52) U.S. Cl. CPC A61K 36/888; A61K 35/742; A61K 35/741; A61K 35/745; A61P 3/04; A23V 2002/00; A23L 33/105; A23L 17/60; A23L 33/135; A23L 33/40; A23L 2/52; A61K 36/02

(57) ABSTRACT

The disclosure relates to methods of treating obesity, abdominal obesity, dyslipidemia, pre-diabetes, or diabetes, or any related disorders thereto; and preventing or attenuating weight gain, including the regain of weight after weight loss, or alleviating or slowing the onset of weight related symptoms and/or disorders. Furthermore, the disclosure teaches methods of administering compositions comprising one or more aquatic plants to shift/optimize the gut microbiome of a patient in need. Additionally, the disclosure provides methods of enhancing the probability of maintaining weight loss and its associated health benefits by sampling a patient's gut microbiome during a period of optimal weight, generally from weight loss, and administering autologous or allogenic microbiota to the patient or to a second patient in order to maintain the weight loss and/or to further prevent or attenuate weight regain.

### CROSS-REFERENCE TO RELATED APPLICATIONS

This application claims the benefit under 35 U.S.C. § 119(e) of U.S. Provisional Patent Application No. 62/726,763, filed Sep. 4, 2018, the contents of which are incorporated herein by reference in their entirety for all purposes.

### FIELD

The present disclosure relates to methods of treating obesity, abdominal obesity, dyslipidemia, pre-diabetes, or diabetes; and preventing or attenuating weight gain after weight loss. Furthermore, the disclosure teaches methods of administering compositions comprising one or more aquatic plants to shift the gut microbiome of a patient or subject in need. Additionally, the disclosure provides methods of maintaining weight loss by sampling a patient's gut microbiome during a period of optimal weight, generally from weight loss, and administering autologous or allogenic microbiota to the patient or to a second patient in order to maintain the weight loss and/or to further prevent or attenuate weight regain.

### BACKGROUND

The human gut microbiota include at least 10¹⁴ bacteria and archaea, containing roughly 1,100 predominant classes, with nearly 160 species per specific class. The unique gut environment has vast prospective for biological interfaces with the host. Duckweeds and algae, among other aquatic plants, possess a relatively high density of dietary fibers, mainly insoluble; and high polyphenol content, including caffeic acid, apigenin, quarcitin, naringenin, kaempferol, among others.

The gut microbiota play a role in the bioavailability and bioactivity of dietary polyphenols. It is likely that most of ingested polyphenols resist degradation in the upper gut, eventually reaching gut bacteria that utilize molecules in the colon, which further degrade the polyphenols into smaller molecules with increased bioavailability and, sometimes, altered biological activity.

The relationship of the gut microbiota with dietary polyphenols may be bi-directional, possibly establishing a key mediator of the health benefits attributed to dietary polyphenols.

In humans, overweight and obese individuals vs normal weight and slim individuals differ in microbiota diversity/profiles/functionality, and these phenotypes can be shifted in germ-free mice. Animal studies and human studies recognize the gut microbiota as a vital causal element to the pathophysiology of obesity. Human body weight loss achieved by lifestyle change, occurs mostly in the first 6 months of intervention (the ‘rapid weight loss phase’), followed by decreased weight loss rate, plateau, or, generally, by weight regain (the regain/maintenance phase).

There is an opportunity to mitigate the rebound effect of weight loss by archiving the gut microbiota of a person that has achieved sustained weight loss on an aquatic plant-based diet for autologous or allogenic administration of the archived microbiota.

### SUMMARY

The disclosure provides methods of altering/optimizing the gut microbiome of a first subject, the method comprising: administering to the first subject, a composition comprising one or more species of a plant rich in dietary fibers and polyphenols. In some embodiments, the plant rich in dietary fibers and polyphenols is duckweed and/or algae. In some embodiments, the method comprises administering the duckweed and/or algae to the first subject until the first subject achieves an optimal/desired weight loss. In some embodiments, the method further comprises obtaining one or more microbiome sample from the gastrointestinal tract of the first subject once the optimal/desired weight loss is achieved. In some embodiments, the method further comprises administering the one or more microbiome sample(s) to the first subject after the optimal/desired weight loss is achieved, and over a period of weight maintenance. In some embodiments, the method further comprises administering the one or more microbiome sample to a second subject.

In some embodiments, a first population of microbes present in the gut microbiome before the administration of the composition increases in abundance/activity after the composition is administered; and/or wherein a second population of microbes present in the gut microbiome before the administration of the composition decreases in abundance/activity after the composition is administered. In some embodiments, a first population of microbes present in the gut microbiome before the administration of the composition increases in abundance/activity after the optimal weight loss is achieved; and/or wherein a second population of microbes present in the gut microbiome before the administration of the composition decreases in abundance/activity after the optimal weight loss is achieved.

In some embodiments, the gut microbiome of the subject comprises a higher proportion of bacteria belonging to at least one genus selected from the group consisting of microbes as Akkermansia, Paraprevotelia, Alistipes, Ruminiclostridium, Bacteroides, Clostridium, Coprococcus, Eubacterium and Lachnoclostridium, after the optimal/desired weight loss is achieved, as compared to before the administration of the composition. In some embodiments, the gut microbiome of the subject comprises a lower proportion of bacteria belonging to at least one genus selected from the group consisting of microbe as Bifidobacterium and Megamonas, after the optimal weight loss is achieved, as compared to before the administration of the composition.

In some embodiments, the gut microbiome of the subject comprises a higher proportion of bacteria belonging to at least one genus selected from the group consisting of microbe as Akkermansia, Paraprevotelia, Eubacterium and Alistipes, at the end of the period of weight maintenance, as compared to before the administration of the composition. In some embodiments, the gut microbiome of the subject comprises a lower proportion of bacteria belonging to at least one genus selected from the group consisting of microbe as Bifidobacterium, at the end of the period of weight maintenance, as compared to before the administration of the composition.

In some embodiments, the one or more species of duckweed belongs to a genus selected from the group consisting of Landoltia, Lemna, Spirodela, Wolffia, and Wolffiella. In some embodiments, the one or more species of duckweed is Wolffia globosa. In some embodiments, the Wolffia globosa is the Mankai varietal. In some embodiments, the one or more species of duckweed/algae are fresh, dehydrated, blended, ground, chopped, frozen, or powdered. In some embodiments, the composition comprises one or more species of algae. In some embodiments, the composition is administered as a tablet, a pill, or a capsule. In some embodiments, the composition comprises an edible product such as a powder, a bar, a smoothie, a yogurt; a dry, frozen or fresh food preparation; or a shake.

In some embodiments, the one or more species of duckweed comprises at least one endophyte selected from the group consisting of microbes as Microbacterium arthrosphaerae, Bacillus niacin, Hymenobacter gelipurpurascens, Arthrobacter agilis, Moraxella_sg, Moraxella osloensis, Terribacillus aidingensis, Terribacillus saccharophilus, Virgibacillus picturae, and Pantoea septica. These specific intrinsic microbes have a potent effect on their environment.

The disclosure provides methods of attenuating the rebound or regain of at least one weight loss-associated metabolic/physical (i.e. waist circumference) parameter after a period of weight loss in a first subject, the method comprising administering to the first subject a composition comprising one or more species of a plant rich in dietary fibers and polyphenols during the period of weight loss. In some embodiments, the plant rich in dietary fibers and polyphenols is duckweed and/or algae. In some embodiments, the method further comprises obtaining one or more microbiome sample from the gastrointestinal tract of the first subject at the end of the period of weight loss. In some embodiments, the method further comprises administering the one or more microbiome sample to the first subject after the end of the period of weight loss, and over a period of weight maintenance. In some embodiments, the method further comprises administering the one or more microbiome sample to a second subject.

In some embodiments, the at least one weight loss-associated metabolic parameter is selected from the level of fasting or post prandial/load plasma insulin, the level of fasting or post prandial/load plasma glucose and waist circumference. In some embodiments, the at least one weight loss-associated metabolic parameter is the level of fasting or post prandial/load plasma insulin. In some embodiments, the rebound % of the level of fasting or post prandial/load plasma insulin in the subject is lower than the rebound % of the level of fasting or post prandial/load plasma insulin in a control subject, wherein the control subject is not administered the one or more species of duckweed/algae over the period of weight loss. In some embodiments, the at least one weight loss-associated metabolic parameter is the level of fasting or post prandial/load plasma glucose. In some embodiments, the rebound % of the level of fasting or post prandial/load plasma glucose in the subject is lower than the rebound % of the level of fasting or post prandial/load plasma glucose in a control subject, wherein the control subject is not administered the one or more species of duckweed over the period of weight loss. In some embodiments, the at least one weight loss-associated metabolic parameter is waist circumference. In some embodiments, the regain % of the waist circumference of the subject is lower than the regain % of the waist circumference of a control subject, wherein the control subject is not administered the one or more species of duckweed over the period of weight loss.

In some embodiments, the one or more species of duckweed belongs to a genus selected from the group consisting of Landoltia, Lemna, Spirodela, Wolffia, and Wolffiella. In some embodiments, the one or more species of duckweed is Wolffia globosa. In some embodiments, the Wolffia globosa is the Mankai varietal. In some embodiments, wherein the one or more species of duckweed are fresh, dehydrated, blended, ground, chopped, frozen, or powdered. In some embodiments, the composition comprises one or more species of algae. In some embodiments, the composition is administered as a tablet, a pill, or a capsule. In some embodiments, the composition comprises an edible product such as a powder, a bar, a smoothie, a yogurt, a fresh/frozen/dry food preparation, or a shake.

In some embodiments, one or more species of duckweed comprises at least one endophyte selected from the group consisting of microbes as Microbacterium arthrosphaerae, Bacillus niacin, Hymenobacter gelipurpurascens, Arthrobacter agilis, Moraxella_sg, Moraxella osloensis, Terribacillus aidingensis, Terribacillus saccharophilus, Virgibacillus picturae, and Pantoea septica.

The disclosure further provides methods for treating obesity or an obesity-related disorder in a first subject, the method comprising administering to the first subject a composition comprising one or more species of a plant rich in dietary fibers and polyphenols. In some embodiments, the plant rich in dietary fibers and polyphenols is duckweed and/or algae. In some embodiments, the method comprises administering the duckweed to the first subject until the subject achieves an optimal weight loss. In some embodiments, the method further comprises obtaining one or more microbiome sample from the gastrointestinal tract of the first subject once the optimal weight loss is achieved. In some embodiments, the method further comprises administering the one or more microbiome sample to the first subject after the optimal weight loss is achieved, and over a period of weight maintenance. In some embodiments, the method further comprises administering the one or more microbiome sample to a second subject.

In some embodiments, the obesity-related disorder is diabetes, pre-diabetes, dyslipidemia, heart disease, stroke, high blood pressure, cancer, gallbladder disease, osteoarthritis, gout, sleep apnea, fatty liver diseases, kidney disease, or any combination thereof; or other excess weight related disease or disorder. In some embodiments, the obesity-related disorder is a breathing problem, such as asthma; a pregnancy problem such as gestational diabetes, preeclampsia, and requiring Cesarean delivery (C-section), requiring longer recovery after birth; mental illness such as clinical depression, anxiety, and other mental disorders; difficulty with physical functioning; and body pain. In some embodiments, the one or more species of duckweed belongs to a genus selected from the group consisting of Landoltia, Lemna, Spirodela, Wolffia, and Wolffiella. In some embodiments, the one or more species of duckweed is Wolffia globosa. In some embodiments, the Wolffia globosa is the Mankai varietal.

In some embodiments, the one or more species of duckweed are fresh, dehydrated, blended, ground, chopped, frozen, or powdered. In some embodiments, the composition comprises one or more species of algae. In some embodiments, the composition is administered as a tablet, a pill, or a capsule. In some embodiments, the composition comprises an edible product such as a powder, a bar, a smoothie, a yogurt, a dry/fresh/frozen food preparation, or a shake.

In some embodiments, one or more species of duckweed comprises at least one endophyte selected from the group consisting of microbes as Microbacterium arthrosphaerae, Bacillus niacin, Hymenobacter gelipurpurascens, Arthrobacter agilis, Moraxella_sg, Moraxella osloensis, Terribacillus aidingensis, Terribacillus saccharophilus, Virgibacillus picturae, and Pantoea septica.

The disclosure also provides methods of attenuating weight regain after a period of weight loss in a subject, the method comprising: (i) administering to the subject a composition comprising one or more species of a plant rich in dietary fibers and polyphenols during the period of weight loss; (ii) obtaining one or more microbiome sample from the gastrointestinal tract of the subject at the end of the period of weight loss; and (iii) administering the one or more microbiome sample to the subject after the period of weight loss, and over a period of weight maintenance. In some embodiments, the plant rich in dietary fibers and polyphenols is duckweed and/or algae.

The disclosure provides methods of attenuating weight regain after a period of weight loss in a subject, the method comprising: (i) obtaining one or more microbiome sample from the gastrointestinal tract of the subject at the end of the period of weight loss; and (ii) administering the one or more microbiome sample to the subject after the period of weight loss, and over a period of weight maintenance. In some embodiments, in step (i), the subject is further administered one or more plant polyphenols. In some embodiments, the percentage of weight lost at the end of the period of weight loss is at least about 0.5%. In some embodiments, the weight regain % in the subject is lower than the weight regain % in a control subject, wherein the control subject is administered the one or more species of duckweed over the period of weight loss, and wherein one or more microbiome sample is not obtained from the gastrointestinal tract of the control subject at the end of the period of weight loss, and not administered to the control subject. In some embodiments, the control subject is a subject who is administrated a placebo during the regain/maintenance phase. In some embodiments, the weight regain % in the subject is lower than the weight regain % in the control subject by at least about 1%. In some embodiments, the weight regain % in the subject is lower than the weight regain % in the control subject by about 30%. In some embodiments, the weight regain % of the subject at the end of the weight maintenance period is less than about 50%.

In some embodiments, the regain % of the waist circumference of the subject is lower than the regain % of the waist circumference of a control subject, wherein the control subject is administered the one or more species of duckweed over the period of weight loss, and wherein one or more microbiome sample is not obtained from the gastrointestinal tract of the control subject at the end of the period of weight loss, and not administered to the control subject. In some embodiments, the control subject is a subject who is administrated a placebo during the regain/maintenance phase. In some embodiments, the regain % of the waist circumference of the subject is lower than the regain % of the waist circumference of a control subject by at least about 1%. In some embodiments, wherein the percentage of waist circumference lost at the end of the period of weight loss is at least 0.5%.

The disclosure also provides methods of attenuating the rebound or regain of at least one weight loss-associated metabolic parameter after a period of weight loss in a subject, the method comprising: (i) administering to the subject a composition comprising one or more species of a plant rich in dietary fibers and polyphenols during the period of weight loss; (ii) obtaining one or more microbiome sample from the gastrointestinal tract of the subject at the end of the period of weight loss; and (iii) administering the one or more microbiome sample to the subject after the end of the period of weight loss, and over a period of weight maintenance. In some embodiments, the plant rich in dietary fibers and polyphenols is duckweed and/or algae.

The disclosure provides methods of attenuating the rebound of at least one weight loss-associated metabolic parameter after a period of weight loss in a subject, the method comprising:
(i) obtaining one or more microbiome sample from the gastrointestinal tract of the subject at the end of the period of weight loss; and (ii) administering the one or more microbiome sample to the subject after the end of the period of weight loss, and over a period of weight maintenance. In some embodiments, the at least one weight loss-associated metabolic parameter is selected from the level of fasting or post prandial/load plasma insulin and level of fasting or post prandial/load plasma glucose.

In some embodiments, the at least one weight loss-associated metabolic parameter is the level of fasting or post prandial/load plasma insulin. In some embodiments, the rebound % of the level of fasting or post prandial/load plasma insulin in the subject is lower than the rebound % of the level of fasting or post prandial/load plasma insulin in a control subject, wherein the control subject is administered the one or more species of duckweed over the period of weight loss, and wherein one or more microbiome sample is not obtained from the gastrointestinal tract of the control subject at the end of the period of weight loss, and not administered to the control subject. In some embodiments, the control subject is a subject who is administrated a placebo during the regain/maintenance phase.

In some embodiments, the at least one weight loss-associated metabolic parameter is the level of fasting or post prandial/load plasma glucose. In some embodiments, the rebound % of the level of fasting or post prandial/load plasma glucose in the subject is lower than the rebound % of the level of fasting or post prandial/load plasma glucose in a control subject, wherein the control subject is administered the one or more species of duckweed over the period of weight loss, and wherein one or more microbiome sample is not obtained from the gastrointestinal tract of the control subject at the end of the period of weight loss, and not administered to the control subject. In some embodiments, the control subject is a subject who is administrated a placebo during the regain/maintenance phase.

The disclosure provides methods of altering/optimizing the gut microbiome of a subject, the method comprising (i) administering to the subject a composition comprising one or more species of one or more species of a plant rich in dietary fibers and polyphenols during a period of weight loss; (ii) obtaining one or more microbiome sample from the gastrointestinal tract of the subject at the end of the period of weight loss; and (iii) administering the one or more microbiome sample to the subject after the end of the period of weight loss, and over a period of weight maintenance. In some embodiments, the plant rich in dietary fibers and polyphenols is duckweed and/or algae.

The disclosure provides methods of altering/optimizing the gut microbiome of a subject after a period of weight loss, the method comprising (i) obtaining one or more microbiome sample from the gastrointestinal tract of the subject at the end of the period of weight loss; and (ii) administering the one or more microbiome sample to the subject after the end of the period of weight loss, and over a period of weight maintenance. In some embodiments, a first population of microbes present in the gut microbiome before step (i) increases in abundance/activity after step (ii); and/or wherein a second population of microbes present in the gut microbiome before step (i) decreases in abundance/activity after step (ii). In some embodiments, a first population of microbes present in the gut microbiome before step (i) increases in abundance/activity after step (iii); and/or wherein a second population of microbes present in the gut microbiome before step (i) decreases in abundance/activity after step (iii).

In some embodiments, the gut microbiome of the subject comprises a higher proportion of bacteria belonging to at least one genus selected from the group consisting of microbes as Akkermansia, Paraprevotelia, Alistipes, Ruminiclostridium, Bacteroides, Clostridium, Coprococcus, Eubacterium and Lachnoclostridium, at the end of the period of weight loss, as compared to the beginning of the period of weight loss. In some embodiments, the gut microbiome of the subject comprises a higher proportion of bacteria belonging to at least one genus selected from the group consisting of microbes as Akkermansia, Paraprevotelia, Eubacterium and Alistipes, at the end of the period of weight maintenance, as compared to the beginning of the period of weight loss.

In some embodiments, the gut microbiome of the subject comprises a lower proportion of bacteria belonging to at least one genus selected from the group consisting of microbes as Bifidobacterium and Megamonas, at the end of the period of weight loss, as compared to the beginning of the period of weight loss. In some embodiments, the gut microbiome of the subject comprises a lower proportion of bacteria belonging the genus as Bifidobacterium, at the end of the period of weight maintenance, as compared to the beginning of the period of weight loss.

The disclosure provides methods of maintaining the gut microbiome of a subject after a period of weight loss, the method comprising: (i) administering to the subject one or more species of a plant rich in dietary fibers and polyphenols during the period of weight loss; (ii) obtaining one or more microbiome sample from the gastrointestinal tract of the subject at the end of the period of weight loss; and (iii) administering the one or more microbiome sample to the subject after the end of the period of weight loss, and over a period of weight maintenance. In some embodiments, the plant rich in dietary fibers and polyphenols is duckweed and/or algae.

In some embodiments, the proportion of bacteria belonging to at least one genus selected from the group consisting of bacteria as Akkermansia, Paraprevotelia, Eubacterium and Alistipes in the gut microbiome of the subject is higher than the proportion of bacteria belonging to at least one genus selected from the group consisting of bacteria as Akkermansia, Paraprevotelia, Eubacterium and Alistipes in the gut microbiome of a control subject during the period of weight maintenance; wherein the control subject is administered the one or more species of duckweed over the period of weight loss, and wherein one or more microbiome sample is not obtained from the gastrointestinal tract of the control subject at the end of the period of weight loss, and the one or more microbiome sample is not administered to the control subject at the end of the period of weight loss. In some embodiments, the control subject is a subject who is administrated a placebo during the regain/maintenance phase.

In some embodiments, the proportion of bacteria belonging the genus Bifidobacterium in the gut microbiome of the subject is lower than the proportion of bacteria belonging the genus Bifidobacterium in the gut microbiome of a control subject, wherein the control subject is administered the one or more species of duckweed over the period of weight loss, and wherein one or more microbiome sample is not obtained from the gastrointestinal tract of the control subject at the end of the period of weight loss, and the one or more microbiome sample is not administered to the control subject at the end of the period of weight loss. In some embodiments, the control subject is a subject who is administrated a placebo during the regain/maintenance phase.

The disclosure provides methods for treating obesity or an obesity-related disorder, after a period of weight loss in a subject, the method comprising: (i) administering to the subject a composition comprising one or more species of a plant rich in dietary fibers and polyphenols during the period of weight loss; (ii) obtaining one or more microbiome sample from the gastrointestinal tract of the subject at the end of the period of weight loss; and (iii) administering the one or more microbiome sample to the subject after the end of the period of weight loss. In some embodiments, the plant rich in dietary fibers and polyphenols is duckweed and/or algae.

In some embodiments, the obesity-related disorder is diabetes, prediabetes, dyslipidemia, heart disease, stroke, high blood pressure, cancer, gallbladder disease, osteoarthritis, gout, sleep apnea, fatty liver diseases, kidney disease, or any combination thereof. In some embodiments, the obesity-related disorder is a breathing problem, such as asthma; a pregnancy problem such as gestational diabetes, preeclampsia, and requiring Cesarean delivery (C-section), requiring longer recovery after birth; mental illness such as clinical depression, anxiety, and other mental disorders; difficulty with physical functioning; and body pain.

The disclosure provides methods for treating obesity, or an obesity-related disorder, comprising administering to a subject a therapeutically effective amount of a composition comprising: (i) obtaining one or more microbiome sample from the gastrointestinal tract of the subject at the end of the period of weight loss; and (ii) administering the one or more microbiome sample to the subject after the end of the period of weight loss.

The disclosure further provides methods for treating an age-related disorders or deterioration in a first subject, the method comprising administering to the first subject a composition comprising one or more species of a plant rich in dietary fibers and polyphenols. In some embodiments, the plant rich in dietary fibers and polyphenols is duckweed and/or algae. In some embodiments, the method comprises administering the duckweed to the first subject until the subject achieves an optimal weight loss. In some embodiments, the method further comprises obtaining one or more microbiome sample from the gastrointestinal tract of the first subject once the optimal weight loss is achieved. In some embodiments, the method further comprises administering the one or more microbiome sample to the first subject after the optimal weight loss is achieved, and over a period of weight maintenance. In some embodiments, the method further comprises administering the one or more microbiome sample to a second subject.

In some embodiments, the one or more species of duckweed belongs to a genus selected from the group consisting of Landoltia, Lemna, Spirodela, Wolffia, and Wolffiella. In some embodiments, the one or more species of duckweed is Wolffia globosa. In some embodiments, the Wolffia globosa is the Mankai varietal.

In some embodiments, the one or more species of duckweed are fresh, dehydrated, blended, ground, chopped, frozen, or powdered. In some embodiments, the composition comprises one or more species of algae. In some embodiments, the composition is administered as a tablet, a pill, or a capsule. In some embodiments, the composition comprises an edible product such as a powder, a bar, a smoothie, a yogurt, a dry/fresh/frozen food preparation, or a shake.

In some embodiments, one or more species of duckweed comprises at least one endophyte selected from the group consisting of microbes as Microbacterium arthrosphaerae, Bacillus niacin, Hymenobacter gelipurpurascens, Arthrobacter agilis, Moraxella_sg, Moraxella osloensis, Terribacillus aidingensis, Terribacillus saccharophilus, Virgibacillus picturae, and Pantoea septica.

The disclosure provides methods for treating an age-related disorder or deterioration, after a period of weight loss in a subject, the method comprising: (i) administering to the subject a composition comprising one or more species of a plant rich in dietary fibers and polyphenols during the period of weight loss; (ii) obtaining one or more microbiome sample from the gastrointestinal tract of the subject at the end of the period of weight loss; and (iii) administering the one or more microbiome sample to the subject after the end of the period of weight loss. In some embodiments, the plant rich in dietary fibers and polyphenols is duckweed and/or algae.

The disclosure provides methods for treating an age-related disorder or deterioration, comprising administering to a subject a therapeutically effective amount of a composition comprising: (i) obtaining one or more microbiome sample from the gastrointestinal tract of the subject at the end of the period of weight loss; and (ii) administering the one or more microbiome sample to the subject after the end of the period of weight loss.

In some embodiments, the age-related disorders or deterioration may affect the physical health, cognition and/or fertility of the subject. In some embodiments, the age-related disorder or deterioration is atherosclerosis, cardiovascular disease, cancer, arthritis, cataracts, osteoporosis, type 2 diabetes, hypertension, Alzheimer's disease, Parkinson's disease, dementia, Chronic Obstructive Pulmonary Disease (COPD), hearing loss, or any combination thereof.

### BRIEF DESCRIPTION OF THE DRAWINGS

FIG. 1 shows an image of several Mankai (Wolffia globosa) cells.

FIG. 2 shows an image of a Mankai (Wolffia globosa) cell, with a scale for indication of its size.

FIG. 3 shows the experimental set up (FIG. 3A) for the Mankai induced fecal transplantation study in mice, and the weight regain in the two different diet groups of mice (FIGS. 3B-3D).

FIG. 4 shows the results of an intraperitoneal glucose tolerance test performed on the two different diet/fecal administered groups of mice.

FIG. 5 shows the human 14-month study design among 90 participants; body weight and blood biomarker changes. FIG. 5A shows experimental design; and FIG. 5B shows body weight changes.

FIG. 6 shows the genus-level bacteria that were significantly changed by weight loss. Values represents log 2 fold change from baseline, by bacteria/module (preliminary analysis).

FIG. 7A shows genus-level bacteria and metabolic pathways (KEGG modules) that were significantly changed by weight loss and maintained by the aFMT treatment (preliminary analysis). FIG. 7B: Principal coordinate analysis (PCoA) of weighted UniFrac distances of microbiome composition. Each sub-plot displays the distances between individual microbiome samples within the distances of the indicated lifestyle intervention, at baseline and 6-months. FIG. 7C: Principal coordinate analysis (PCoA) of Bray-Curtis distances of microbiome composition, each sub-plot displays the distances between individual microbiome samples within the indicated lifestyle intervention and treatment group, at 6 and 14-months.

FIG. 8 shows Aerobic Total count of crushed and non-crushed plant after surface disinfection. Post plant surface disinfection by elevated H2O2 doses (0%-7%) a significant reduction in aerobic total bacterial count was observed in non-crushed plant in correlation with dose increase. However, in crushed plant, the colony forming units were significantly higher and only slightly decreased as H2O2 doses increased.

FIG. 9 shows B12 Vitamin levels in Mankai plant cultures under different microbial load.

FIG. 10 shows the effect of Mankai extract on endophyte growth dynamic.

FIG. 11 shows the effect of Mankai on the medium microbial levels.

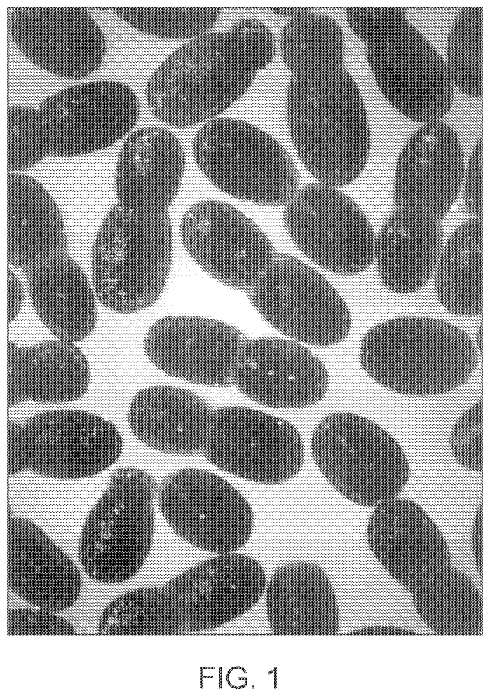

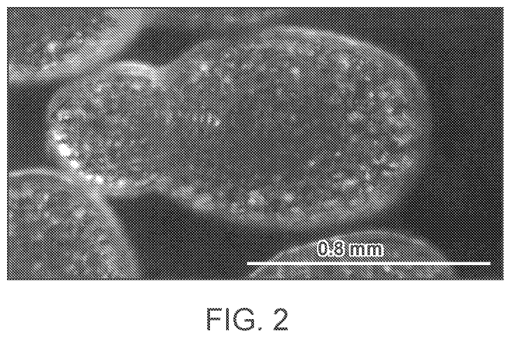

### DETAILED DESCRIPTION

### Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein.

In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, the terms “about” or “approximately” when preceding a numerical value indicates the value plus or minus a range of 10%.

As used herein, “carrier”, “acceptable carrier”, or “pharmaceutical carrier” refers to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed. In some aspects, gelling agents are employed as carriers. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Hardee and Baggo (1998. Development and Formulation of Veterinary Dosage Forms. 2^{nd} Ed. CRC Press. 504 pg.); E. W. Martin (1970. Remington' s Pharmaceutical Sciences. 17^{th} Ed. Mack Pub. Co.); and Blaser et al. (US Publication US20110280840A1).

As used herein, “microbiome” refers to the collection of microorganisms that inhabit the digestive tract or gastrointestinal (GI) tract of an animal (including the GI tract of a human) and the microorganism's physical environment (i.e. the microbiome has a biotic and abiotic component). The microbiome is fluid and may be modulated by numerous naturally occurring and artificial conditions (e.g., change in diet, disease, antimicrobial agents, influx of additional microorganisms, etc.). The modulation or optimization or alteration or shifting of the gut microbiome achieved via administration of the compositions of the present disclosure, can take the form of: (a) changing the diversity; i.e., increasing or decreasing a particular Family, Genus, Species, or functional grouping of microbes (i.e. alteration of the biotic component of the microbiome); (b) increasing or decreasing ratios of a particular Family, Genus, Species, or functional grouping of microbes; (c) increasing or decreasing polyphenols, and proteins (i.e. alteration of the abiotic components of the microbiome) and/or (d) changing the quality of the microbiome; i.e., increasing the proportion of microbes associated with weight loss associated health benefits, and/or decreasing the proportion of microbes associated with weight gain-associated adverse health effects.

As used herein, “probiotic” refers to a substantially pure microbe (i.e., a single isolate) or a mixture of desired microbes; and may also include any additional components that can be administered for restoring microbiota. Probiotics or microbial inoculant compositions of the disclosure may be administered with an agent to allow the microbes to survive the environment of the gastrointestinal tract, i.e., to resist low pH and to grow in the gastrointestinal environment.

As used herein, “prebiotic” refers to an agent that increases the number and/or activity of one or more desired microbes. Non-limiting examples of prebiotics that may be useful in the methods of the present disclosure include fructooligosaccharides (e.g., oligofructose, inulin, inulin-type fructans), galactooligosaccharides, glucans (β-glucans), amino acids, alcohols, dietary fibers (soluble or insoluble), and mixtures thereof. See Ramirez-Farias et al. (2008. Br. J. Nutr. 4:1-10) and Pool-Zobel and Sauer (2007. J. Nutr. 137:2580-2584 and supplemental).

As used herein “shelf-stable” refers to a functional attribute and new utility acquired by the microbes formulated according to the disclosure, which enable said microbes to exist in a useful/active state outside of their natural environment in the GI tract (i.e. a markedly different characteristic). Thus, shelf-stable is a functional attribute created by the formulations/compositions of the disclosure and denoting that the microbe formulated into a shelf-stable composition can exist outside the GI tract and under ambient conditions for a period of time that can be determined depending upon the particular formulation utilized, but in general means that the microbes can be formulated to exist in a composition that is stable under ambient conditions for at least a few days and generally at least one week. Accordingly, a “shelf-stable autologous sample” is a composition comprising one or more microbes (without passage or including many passages) from the GI of the patient, said microbes formulated in a composition, such that the composition is stable under ambient conditions for at least one week, meaning that the microbes comprised in the composition are viable or replicating in the GI tract.

As used herein, an “aquatic plant” is a plant that has adapted to living in an aquatic environment (freshwater, brackish water, or saltwater). Aquatic plants grow in or near water, and are either emergent, submergent, or floating. Aquatic plants can only grow in water or soil that is permanently saturated with water. Aquatic plants are commonly referred to as macrophytes or hydrophytes.

As used herein, “autologous” refers to cells, tissues, samples, compositions obtained from the same individual. An autologous microbiome composition administered to a patient is a microbiome composition that was obtained, at least in part, for the same patient in which it was ultimately administered. An autologous fecal sample is one in which is obtained from a patient and the microbes in the fecal sample are isolated and administered to the patient. These isolated microbes are autologous because there were from the fecal sample of the patient.

As used herein, “abdominal obesity” refers to a person having a waist circumference of greater than 101 cm for men and 66 cm for woman.

As used herein, “pre-diabetes” or “diabetes” refers to a person having a serum fasting glucose measurement of greater than 110 mg/dl.

As used herein, “dyslipedema” refers to a man having an HDL measurement of less than 40 mg/dl or a woman having an HDL measurement of less than 50 mg/dl; and/or a triglyceride measurement of greater than 150 mg/dl.

As used herein “adiposity” or “obesity” are used interchangeably to mean a medical condition in which a person has accumulated excess body fat to the extent that it may have a negative effect on heath. People are generally considered obese when their body mass index (BMI), a measure obtained by dividing a person's weight by the square of a person's height, is greater than 25 kg/m². As used herein, “obesity-related disorder” refers to a health condition that is associated with, correlated with and/or caused by the presence of obesity. In some embodiments, obesity increases the risk of developing certain diseases. Non-limiting examples of obesity-related disorders include diabetes, prediabetes, dyslipidemia, heart disease, stroke, high blood pressure, cancer, gallbladder disease, osteoarthritis, gout, sleep apnea, fatty liver diseases, kidney disease, a breathing problem, such as asthma; a pregnancy problem such as gestational diabetes, preeclampsia, and requiring Cesarean delivery (C-section), requiring longer recovery after birth; mental illness such as clinical depression, anxiety, and other mental disorders; difficulty with physical functioning; body pain, or any combination thereof.

As used herein, “weight-related disorder” refers to a health condition that is associated with, correlated with and/or caused by the weight of the subject. In some embodiments, increase in weight increases the risk of developing certain diseases. Non-limiting examples of weight-related disorders include diabetes, prediabetes, dyslipidemia, heart disease, stroke, high blood pressure, cancer, gallbladder disease, osteoarthritis, gout, sleep apnea, fatty liver diseases, kidney disease, a breathing problem, such as asthma; a pregnancy problem such as gestational diabetes, preeclampsia, and requiring Cesarean delivery (C-section), requiring longer recovery after birth; mental illness such as clinical depression, anxiety, and other mental disorders; difficulty with physical functioning; body pain, or any combination thereof.

As used herein, “age-related disorder” refers to a health condition that is associated with, correlated with and/or caused by the age of the subject. In some embodiments, the risk of developing age-related disorders increases with increasing age of the subject. In some embodiments, an age-related disorder is seen with increasing frequency with increasing senescence. Non-limiting examples of age-related disorders include atherosclerosis, cardiovascular disease, cancer, arthritis, cataracts, osteoporosis, type 2 diabetes, hypertension, Alzheimer's disease, Parkinson's disease, dementia, Chronic Obstructive Pulmonary Disease (COPD), hearing loss, or any combination thereof.

As used herein “body mass index” or “BMI” refer to a value derived from the weight and height of a person. BMI is a measure obtained by dividing a person's weight by the square of a person's height, expressed as kg/m². The scale of BMI values are used to qualitatively indicate a condition of underweight (less than 18.5 kg/m²), normal weight (18.5 to 25 kg/m²), overweight (25 to 30 kg/m²), and obese (greater than 25 kg/m²).

As used herein “optimal weight loss” is used to indicate a weight loss of at least 1%.

As used herein “optimal sustained weight loss” is used to indicate a weight loss of at least 1% for a sustained period of time.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Markers further include polynucleotide sequences encoding 16S or 18S rRNA, and internal transcribed spacer (ITS) sequences, which are sequences found between small-subunit and large-subunit rRNA genes that have proven to be especially useful in elucidating relationships or distinctions among when compared against one another.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45). The 16S rRNA encoded by 16S rDNA can be utilized to map the relative and/or absolute composition of the gut microbiota at any given time point. The comparative analysis of 16S rRNA gene sequences enables the establishment of taxonomic thresholds that are useful not only for the classification of cultured microorganisms but also for the classification of the many environmental sequences.

As used herein, the term “trait” refers to a characteristic or phenotype. For example, in the context of some embodiments of the present disclosure; desirable traits may include, but not limited to: reduction in obesity, increase in glycemic control, an increase in favorable cardiometabolic outcomes; and any combination thereof; wherein said increase or reduction is determined by comparing against a patient or subject not having been (1) administered an aquatic plant-based diet and/or an autologous microbiome dose(s) at one or more points in time after achieving an optimal sustained weight loss.

As used herein, the term “microbiome modulator” references an agent that is capable of altering/optimizing the gut microbiota of a patent or subject administered a composition of the present disclosure. Microbiome modulators shift the gastrointestinal microbiota, and their environment, in specific ways to achieve improved health traits, including weight loss, sustained weight loss, and decreasing BMI. The change in the microbial community may involve an increase or decrease in one or more species of the microbial community. In the present application, microbiome modulators include aquatic plants and algae, and components thereof, including polyphenols and polysaccharides such as dietary fibers (β-glucan and inulin, among others). Microbiome modulators also include other microbes that can shift the composition of the gut microbiota upon administration.

As used herein, the weight of the subject at the beginning of the weight loss period is referred to herein as the “original weight” of the subject.

A “period of weight loss” (interchangeably referred to herein as “rapid weight loss period”) is defined as a period time over which the subject is subjected to one or more lifestyle interventions. The lifestyle intervention may comprise any activity that is known, or is predicted, to induce weight loss, such as, for example, physical activity and/or any dietary restriction. For instance, the diet of a subject during the weight loss period may be a diet that is low in carbohydrates, rich in plants, rich in polyphenols, low in processed meat, or a combination thereof. In some embodiments, the physical activity of a subject during the weight loss period may comprise moderate-intensity physical activity. In some embodiments, the physical activity may comprise aerobic activity. The length of the period of weight loss is not limited, and may be about 1 day to about 1 year, for example about 1 day, about 3 days, about 1 week, about 3 weeks, about 1 month, about 3 months, about 6 months, or about 9 months, including all values and subranges that lie therebetween.

The weight of the subject at the end of the weight loss period is referred to herein as the “reduced weight” of the subject. In some embodiments, the reduced weight of the subject is less than the original weight of the subject.

As used herein, “weight loss” refers to the difference between the reduced weight and the original weight, wherein the reduced weight is lower than the original weight.

As used herein, “weight loss percentage” is calculated by the following formula:

(Original weight−Reduced weight)/(Original weight)*100.

In some embodiments, the reduced weight of the subject is less than the original weight of the subject by at least 0.5%. For example, the reduced weight of the subject may be less than the original weight of the subject by about 1%, about 1.5%, about 2%, about 2.5%, about 3%, about 3.5%, about 4%, about 4.5%, about 5%, about 6%, about 7%, about 8%, about 9%, or about 10%, including all values and subranges that lie therebetween.

In some embodiments, the weight loss percentage is at least 0.5%. For example, the weight loss percentage may be about 1%, about 1.5%, about 2%, about 2.5%, about 3%, about 3.5%, about 4%, about 4.5%, about 5%, about 6%, about 7%, about 8%, about 9%, or about 10%, including all values and subranges that lie therebetween.

A period of weight maintenance (interchangeably referred to herein as weight regain period) is defined herein as a period during which the subject is no longer subjected to the lifestyle intervention of the weight loss period. In some embodiments, the weight maintenance period immediately follows the weight loss period. The weight of the subject at the end of the weight regain period is referred to herein as the “adjusted weight” of the subject. The length of the period of weight regain is not limited, and may be about 1 day to several years, for example about 1 day, about 3 days, about 1 week, about 3 weeks, about 1 month, about 2 months, about 3 months, about 6 months, about 8 months, about 9 months, about 12 months, about 14 months including all values and subranges that lie therebetween.

In some embodiments, the adjusted weight is more than the reduced weight. In some embodiments, the adjusted weight is more than the reduced weight by at least about 0.5 kgs.

As used herein, “weight regain” refers to the difference between the adjusted weight and the reduced weight, wherein the adjusted weight is higher than the reduced weight.

As used herein, “weight regain percentage” is calculated by the following formula:

(Adjusted weight−Reduced weight)/(Reduced weight−Original weight)*100.

“Weight loss-associated metabolic parameter” (or “weight gain-associated metabolic parameter) refers to any metabolic parameter, the value of which is altered, or expected to be altered, by loss and/or gain of weight. The term “metabolic parameter” is not limited and may refer to any parameter related to metabolism, including any biochemical process that occurs within the subject. Non limiting examples of weight loss associated metabolic parameters include level of high density lipoprotein (HDL) cholesterol, level of low density lipoprotein (LDL) cholesterol, level of leptin, level of inflammatory markers such as, IL-6, fasting lipid profile, glycemic control, level of fasting or post prandial/load plasma glucose, level of fasting or post prandial/load plasma insulin, insulin resistance, and level of high-sensitivity C reactive protein.

As used herein, the regain of a weight loss-associated parameter (WLP) refers to the difference between the value of the weight loss-associated parameter at the end of the weight maintenance period and the value of the weight loss-associated parameter at the end of the weight loss period. As used herein, “regain percentage” is calculated by the following formula:

(WLP at end of weight maintenance period−WLP at end of weight loss period)/(WLP at end of weight loss period−WLP at the beginning of weight loss period)*100.

### Dietary Fibers

In some aspects, dietary fibers, such as β-glucan and inulin, can be isolated from various sources such as oat, barley, wheat, baker's yeast, and some fungi.

Dietary fibers may be fermented in the lower GI tract, resulting in an increased viscosity of the surrounding digesta. High digesta viscosities delays gastric emptying and slows digestion and absorption of nutrients. The bioactivities of β-glucan and inulin in the GI tract may account for an increase in post-prandial satiety in response to a standard meal, reduce glucose and insulin response to a breakfast meal, reduce the glycemic index, increase fasting peptide YY and glucagon-like peptide-1, decrease ghrelin levels, reduce body weight, and increase insulin sensitivity in humans. See Greenway et al. (2014 Beneficial Microbes. 5(1):29-32).

### Polyphenols

Polyphenolics or polyphenols are secondary metabolites of plants and are generally involved in defense against ultraviolet radiation or pathogens. In edible matter, polyphenols may contribute to the bitterness, astringency, color, flavor, odor, and oxidative stability. Polyphenol compounds are naturally occurring compounds found largely in the fruits, vegetables, and cereals; however, polyphenols can be found throughout a variety of plant structures. See Pandey and Rizvi (2009. Oxid Med Cell Longev. Nov-Dec. 2(5):270-278).

Polyphenols are classified on the basis of the number of phenol rings that they contain and of the structural elements that bind these rings to one another. They are broadly divided into four classes: phenolic acids, flavonoids, stilbenes, and lignans. Phenolic acids are further divided into hydroxyl benzoic and hydroxyl cinnamic acids. Non-limiting examples of phenolic acids include caffeic acid, gallic acid, and ferulic acid. Flavonoids share a common basic structure consisting of two aromatic rings which are bound together by three carbon atoms that form an oxygenated heterocycle. One ring usually arises from a molecule of resorcinol, and the other ring is derived from the shikimate pathway. Non-limiting examples of flavonoids include cyanidin, malvidin, glycitein, genistein, catechin, and leucoanthocyanidin. Stilbenes contain two phenyl moieties connected by a two-carbon methylene bridge. Most stilbenes in plants act as antifungal phytoalexins, compounds that are synthesized in response to infection or injury. Non-limiting examples of stilbenes include resveratrol, viniferins, pterostilbene, and pinosylvin. Lignans are diphenolic compounds that contain a 2,3-dibenzylbutane structure that is formed by the dimerization of two cinnamic acid residues. Non-limiting examples of lignans include secoisolariciresinol, isolariciresinol, matairesinol, and lariciresinol. See Pandey and Rizvi.

In some aspects, one or more exogenous polyphenol is administered with the one or more aquatic plant or algae. In some aspects, a composition comprising the one or more aquatic plant or algae further comprise one or more exogenous polyphenols. In some aspects, one or more exogenous phenolic acid, flavonoid, stilbene, and/or lignin is administered with the one or more aquatic plant or algae. In some aspects, a composition comprising the one or more aquatic plant or algae further comprise one or more exogenous phenolic acid, flavonoid, stilbene, and/or lignin.

### Subject/Patient

In some aspects the subject or patient is a male. In some aspects, the subject or patient is a female. In some aspects, the female is pregnant. In some aspects, the subject or patient is a senior greater than 65 years of age. In some aspects, the subject or patient is a senior greater than 75 years of age. In some aspects, the subject or patient is a child less than 18 years of age. In some aspects, the subject or patient is a child less than 16 years of age.

### Optimal Weight Loss

As used herein “optimal weight loss” is used to indicate a weight loss of at least 1%. In some embodiments, the weight loss is maintained for a sustained period of time.

In some aspects, the optimal weight loss is sustained for a period of time of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some aspects, the optimal weight loss is sustained for a period of time of at least 1, 2, 3, 4, 5, 6, 7, 8, 9, 10, 11, or 12 months. In some aspects, the optimal weight loss is sustained for a period of time of at least 1 to 2, 1 to 3, 1 to 4, 1 to 5, 1 to 6, 1 to 7, 1 to 8, 1 to 9, 1 to 10, 1 to 11, 1 to 12, 2 to 3, 2 to 4, 2 to 5, 2 to 6, 2 to 7, 2 to 8, 2 to 9, 2 to 10, 2 to 11, 2 to 12, 3 to 4, 3 to 5, 3 to 6, 3 to 7, 3 to 8, 3 to 9, 3 to 10, 3 to 11, 3 to 12, 4 to 5, 4 to 6, 4 to 7, 4 to 8, 4 to 9, 4 to 10, 4 to 11, 4 to 12, 5 to 6, 5 to 7, 5 to 8, 5 to 9, 5 to 10, 5 to 11, 5 to 12, 6 to 7, 6 to 8, 6 to 9, 6 to 10, 6 to 11, 6-12, 7 to 8, 7 to 9, 7 to 10, 7 to 11, 7 to 12, 8 to 9, 8 to 10, 8 to 11, 8 to 12, 9 to 10, 9 to 11, 9 to 12, 10 to 11, 10 to 12, or 11 to 12 months.

In some aspects, the optimal weight loss is sustained for a period of time of at least about 1, about 2, about 3, about 4, about 5, about 6, about 7, about 8, about 9, about 10, about 11, or about 12 months. In some aspects, the optimal weight loss is sustained for a period of time of at least about 1 to 2, about 1 to 3, about 1 to 4, about 1 to 5, about 1 to 6, about 1 to 7, about 1 to 8, about 1 to 9, about 1 to 10, about 1 to 11, about 1 to 12, about 2 to 3, about 2 to 4, about 2 to 5, about 2 to 6, about 2 to 7, about 2 to 8, about 2 to 9, about 2 to 10, about 2 to 11, about 2 to 12, about 3 to 4, about 3 to 5, about 3 to 6, about 3 to 7, about 3 to 8, about 3 to 9, about 3 to 10, about 3 to 11, about 3 to 12, about 4 to 5, about 4 to 6, about 4 to 7, about 4 to 8, about 4 to 9, about 4 to 10, about 4 to 11, about 4 to 12, about 5 to 6, about 5 to 7, about 5 to 8, about 5 to 9, about 5 to 10, about 5 to 11, about 5 to 12, about 6 to 7, about 6 to 8, about 6 to 9, about 6 to 10, about 6 to 11, about 6 to 12, about 7 to 8, about 7 to 9, about 7 to 10, about 7 to 11, about 7 to 12, about 8 to 9, about 8 to 10, about 8 to 11, about 8 to 12, about 9 to 10, about 9 to 11, about 9 to 12, about 10 to 11, about 10 to 12, or about 11 to 12 months.

### Body Mass Index (BMI)

In some aspects, the compositions of the present disclosure administered to a subject or patient results in weight loss such that the subject or patient possesses a BMI of 18.5, 19.0, 19.5, 20.0, 20.5, 21.0, 21.5, 22.0, 22.5, 23.0, 23.5, 24.0, 24.5, or 25.0. In some aspects, the subject or patient has achieved a BMI of about 18.5, about 19.0, about 19.5, about 20.0, about 20.5, about 21.0, about 21.5, about 22.0, about 22.5, about 23.0, about 23.5, about 24.0, about 24.5, or about 25.0 kg/m².

In some aspects, the compositions of the present disclosure administered to a subject or patient results in weight loss such that the subject or patient possesses a BMI of 18.5 to 19.0, 18.5 to 19.5, 18.5 to 20.0, 18.5 to 20.5, 18.5 to 21.0, 18.5 to 21.5, 18.5 to 22.0, 18.5 to 22.5, 18.5 to 23.0, 18.5 to 23.5, 18.5 to 24.0, 18.5 to 24.5, 18.5 to 25.0, 19.0 to 19.5, 19.0 to 20.0, 19.0 to 20.5, 19.0 to 21.0, 19.0 to 21.5, 19.0 to 22.0, 19.0 to 22.5, 19.0 to 23.0, 19.0 to 23.5, 19.0 to 24.0, 19.0 to 24.5, 19.0 to 25.0, 19.5 to 20.0, 19.5 to 20.5, 19.5 to 21.0, 19.5 to 21.5, 19.5 to 22.0, 19.5 to 22.5, 19.5 to 23.0, 19.5 to 23.5, 19.5 to 24.0, 19.5 to 24.5, 19.5 to 25.0, 20.0 to 20.5, 20.0 to 21.0, 20.0 to 21.5, 20.0 to 22.0, 20.0 to 22.5, 20.0 to 23.0, 20.0 to 23.5, 20.0 to 24.0, 20.0 to 24.5, 20.0 to 25.0, 20.5 to 21.0, 20.5 to 21.5, 20.5 to 22.0, 20.5 to 22.5, 20.5 to 23.0, 20.5 to 23.5, 20.5 to 24.0, 20.5 to 24.5, 20.5 to 25.0, 21.0 to 21.5, 21.0 to 22.0, 21.0 to 22.5, 21.0 to 23.0, 21.0 to 23.5, 21.0 to 24.0, 21.0 to 24.5, 21.0 to 25.0, 21.5 to 22.0, 21.5 to 22.5, 21.5 to 23.0, 21.5 to 23.5, 21.5 to 24.0, 21.5 to 24.5, 21.5 to 25.0, 22.0 to 22.5, 22.0 to 23.0, 22.0 to 23.5, 22.0 to 24.0, 22.0 to 24.5, 22.0 to 25.0, 22.5 to 23.0, 22.5 to 23.5, 22.5 to 24.0, 22.5 to 24.5, 22.5 to 25.0, 23.0 to 23.5, 23.0 to 24.0, 23.0 to 24.5, 23.0 to 25.0, 23.5 to 24.0, 23.5 to 24.5, 23.5 to 25.0, 24.0 to 24.5, 24.0 to 25.0, or 24.5 to 25.0 kg/m2.

In some aspects, the compositions of the present disclosure administered to a subject or patient results in weight loss such that the subject or patient possesses a BMI of about 18.5 to 19.0, about 18.5 to 19.5, about 18.5 to 20.0, about 18.5 to 20.5, about 18.5 to 21.0, about 18.5 to 21.5, about 18.5 to 22.0, about 18.5 to 22.5, about 18.5 to 23.0, about 18.5 to 23.5, about 18.5 to 24.0, about 18.5 to 24.5, about 18.5 to 25.0, about 19.0 to 19.5, about 19.0 to 20.0, about 19.0 to 20.5, about 19.0 to 21.0, about 19.0 to 21.5, about 19.0 to 22.0, about 19.0 to 22.5, about 19.0 to 23.0, about 19.0 to 23.5, about 19.0 to 24.0, about 19.0 to 24.5, about 19.0 to 25.0, about 19.5 to 20.0, about 19.5 to 20.5, about 19.5 to 21.0, about 19.5 to 21.5, about 19.5 to 22.0, about 19.5 to 22.5, about 19.5 to 23.0, about 19.5 to 23.5, about 19.5 to 24.0, about 19.5 to 24.5, about 19.5 to 25.0, about 20.0 to 20.5, about 20.0 to 21.0, about 20.0 to 21.5, about 20.0 to 22.0, about 20.0 to 22.5, about 20.0 to 23.0, about 20.0 to 23.5, about 20.0 to 24.0, about 20.0 to 24.5, about 20.0 to 25.0, about 20.5 to 21.0, about 20.5 to 21.5, about 20.5 to 22.0, about 20.5 to 22.5, about 20.5 to 23.0, about 20.5 to 23.5, about 20.5 to 24.0, about 20.5 to 24.5, about 20.5 to 25.0, about 21.0 to 21.5, about 21.0 to 22.0, about 21.0 to 22.5, about 21.0 to 23.0, about 21.0 to 23.5, about 21.0 to 24.0, about 21.0 to 24.5, about 21.0 to 25.0, about 21.5 to 22.0, about 21.5 to 22.5, about 21.5 to 23.0, about 21.5 to 23.5, about 21.5 to 24.0, about 21.5 to 24.5, about 21.5 to 25.0, about 22.0 to 22.5, about 22.0 to 23.0, about 22.0 to 23.5, about 22.0 to 24.0, about 22.0 to 24.5, about 22.0 to 25.0, about 22.5 to 23.0, about 22.5 to 23.5, about 22.5 to 24.0, about 22.5 to 24.5, about 22.5 to 25.0, about 23.0 to 23.5, about 23.0 to 24.0, about 23.0 to 24.5, about 23.0 to 25.0, about 23.5 to 24.0, about 23.5 to 24.5, about 23.5 to 25.0, about 24.0 to 24.5, about 24.0 to 25.0, or about 24.5 to 25.0 kg/m².

### Treating Obesity with an Aquatic Plant and/or Algae Composition

In some aspects, an aquatic plant and/or algae composition is administered to a patient or a subject to treat obesity. In some aspects, an aquatic plant and/or algae composition is administered to a patient or a subject to decrease body weight. In some aspects, an aquatic plant and/or algae composition is administered to a patient or a subject to shift the microbiome of the patient or subject. In some aspects, an aquatic plant and/or algae composition is administered to a patient or a subject to prevent weight regain post-weight loss. In some aspects, an aquatic plant and/or algae composition is administered to a patient or a subject to improve cardiometabolic health. In some aspects, an aquatic plant and/or algae composition is administered to a patient or a subject to improve serum lipid and or glucose profiles. In some aspects, the aquatic plant and/or algae composition sample is later reintroduced to the same patient or subject from which it was collected as a means for slowing weight regain, as compared to a patient or subject receiving a placebo control.

### I. Aquatic Plants and Algae

In some aspects, one or more aquatic plants and/or algae are administered to patients or subjects in need. In some aspects, the algae are microalgae. In some aspects, the algae are macroalgae.

In some aspects, algae species may be selected from the following phyla: Cyanobacteria, Cyanophyta, Prochlorophyta, Rhodophyta, Glaucophyta, Chlorophyta, Dinophyta, Cryptophyta, Chrysophyta, Prymnesiophyta (Haptophyta), Bacillariophyta, Xanthophyta, Eustigmatophyta, Rhaphidophyta, and Phaeophyta.

In some aspects, algae in multicellular or filamentous forms, such as seaweeds or macroalgae, many of which belong to the phyla Phaeophyta or Rhodophyta, are contemplated. In some aspects algae that are microscopic, are contemplated. Many such microalgae occur in unicellular or colonial form.

In some aspects, algae species may comprise cyanobacteria (also known as blue-green algae) from one or more of the following taxonomic groups: Chroococcales, Nostocales, Oscillatoriales, Pseudanabaenales, Synechococcales, and Synechococcophycideae. Non-limiting examples include Gleocapsa, Pseudoanabaena, Oscillatoria, Microcystis, Synechococcus and Arthrospira species.

In some aspects, algae species may comprising one or more of the following taxonomic classes: Euglenophyceae, Dinophyceae, and Ebriophyceae. Non-limiting examples include Euglena species and the freshwater or marine dinoflagellates.

In some aspects, algae species may be green algae selected from one or more of the following taxonomic classes: Micromonadophyceae, Charophyceae, Ulvophyceae and Chlorophyceae. Non-limiting examples include species of Borodinella, Chlorella (e.g., C. ellipsoidea), Chlamydomonas, Dunaliella (e.g., D. salina, D. bardawil), Franceia, Haematococcus, Oocystis (e.g., O. parva, O. pustilla), Scenedesmus, Stichococcus, Ankistrodesmus (e.g., A. falcatus), Chlorococcum, Monoraphidium, Nannochloris and Botryococcus (e.g., B. braunii).

In some aspects, algae species may be golden-brown algae selected from one or more of the following taxonomic classes: Chrysophyceae and Synurophyceae. Non-limiting examples include Boekelovia species (e.g. B. hooglandii) and Ochromonas species.

In some aspects, algae species may be freshwater, brackish, marine, or briny diatoms selected from one or more of the following taxonomic classes: Bacillariophyceae, Coscinodiscophyceae, and Fragilariophyceae. Non-limiting examples include Achnanthes (e.g., A. orientalis), Amphora (e.g., A. coffeiformis strains, A. delicatissima), Amphiprora (e.g., A. hyaline), Amphipleura, Chaetoceros (e.g., C. muelleri, C. gracilis), Caloneis, Camphylodiscus, Cyclotella (e.g., C. cryptica, C. meneghiniana), Cricosphaera, Cymbella, Diploneis, Entomoneis, Fragilaria, Hantschia, Gyrosigma, Melosira, Navicula (e.g., N. acceptata, N. biskanterae, N. pseudotenelloides, N. saprophila), Nitzschia (e.g., N. dissipata, N. communis, N. inconspicua, N. pusilla strains, N. microcephala, N. intermedia, N. hantzschiana, N. alexandrina, N. quadrangula), Phaeodactylum (e.g., P. tricornutum), Pleurosigma, Pleurochrysis (e.g., P. carterae, P. dentata), Selenastrum, Surirella and Thalassiosira (e.g., T. weissflogii).

In some aspects, algae species may be plankton including microalgae that are characteristically small with a diameter in the range of 1 to 10 μm, or 2 to 4 μm. Many of such algae are members of Eustigmatophyta, such as but not limited to Nannochloropsis species (e.g. N. salina).

In some aspects, algae may be selected from one or more algae from the following groups: Coelastrum, Chlorosarcina, Micractinium, Porphyridium, Nostoc, Closterium, Elakatothrix, Cyanosarcina, Trachelamonas, Kirchneriella, Carteria, Crytomonas, Chlamydamonas, Planktothrix, Anabaena, Hymenomonas, Isochrysis, Pavlova, Monodus, Monallanthus, Platymonas, Pyramimonas, Stephanodiscus, Chroococcus, Staurastrum, Netrium, and Tetraselmis.

In some aspects, aquatic plants may be species of duckweed selected from one or more of the following genera: Landoltia, Lemna, Spirodela, Wolffia, and Wolffiella.

In some aspects, species of duckweed may be selected from the following: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdiviana); genus Spirodela (S. intermedia, S. polyrrhiza, S. punctata); genus Wolffia (Wa. angusta, Wa. arrhiza, Wa. australina, Wa. borealis, Wa. brasiliensis, Wa. columbiana, Wa. elongata, Wa. globosa, Wa. microscopica, Wa. neglecta) and genus Wolfiella (Wl. caudata, Wl. denticulata, Wl. gladiata, Wl. hyalina, Wl. lingulata, Wl. repunda, Wl. rotunda, and Wl. neotropica). Any other genera or species of Lemnaceae, if they exist, are also aspects of the present invention. Lemna species can be classified using the taxonomic scheme described by Landolt, Biosystematic Investigation on the Family of Duckweeds: The family of Lemnaceae—A Monograph Study. Geobatanischen Institut ETH, Stiftung Rubel, Zurich (1986)).

In some aspects, Wolffia globosa is selected. In some aspects, the Wolffia globosa Mankai varietal is selected.

In some aspects, species of aquatic plants may be selected from the following groups: Albidella, Aldrovanda vesiculosa, Alisma wahlenbergii, Alismataceae, Alternanthera philoxeroides, Alternanthera reineckii, Althenia, Ammannia gracilis, Ammannia senegalensis, Anubiadeae, Anubias afzelii, Anubias barteri, Anubias barteri var. angustifolia, Anubias barteri var. caladiifolia, Anubias barteri var. glabra, Anubias barteri var. nana, Anubias gigantean, Anubias gilletii, Anubias gracilis, Anubias hastifolia, Anubias heterophylla, Anubias pynaertii, Aponogeton, Aponogeton lakhonensis, Aponogeton longiplumulosus, Aponogeton madagascariensis, Aponogeton undulates, Appertiella, Astonia, Barclaya, Blyxa, Butomus umbellatus, Callitriche stagnalis, Carex lasiocarpa, Carex limosa, Carex nebrascensis, Ceratopteris richardii, Ceratopteris thalictroides, Chamaegigas, Commelina fluviatilis, Crassula aquatica, Crinum thaianum, Cryptocoryne affinis, Cryptocoryne aponogetifolia, Cryptocoryne auriculate, Cryptocoryne beckettii, Cryptocoryne cruddasiana, Cryptocoryne dewitii, Cryptocoryne mekongensis, Cryptocoryne spiralis, Cryptocoryne undulata, Cryptocoryne walker, Cryptocoryne wendtii, Cryptocoryne x willisii, Cryptocoryneae, and Cyperus papyrus.

In some aspects, species of aquatic plants may be selected from the following groups: Echinodorus emersus, Echinodorus reptilis, Eleocharis, Glossostigma cleistanthum, Helanthium, Helanthium bolivianum, Helanthium tenellum, Helanthium zombiense, Helobiae, Hemianthus callitrichoides, Hemianthus micranthemoides, Hottonia inflata, Hottonia palustris, Hydrilla, Hydrocharis, Hydrocharitaceae, Hydrocotyle umbellate, Hygrophila corymbose, Hygrophila costata, Hygrophila difformis, Hygrophila madurensis, Hygrophila polysperma, Isoetes appalachiana, Isoetid, Juncaginaceae, Lagarosiphon, Lemna, Lemnoideae, Lilaea scilloides, Lilaeopsis brasiliensis, Lilaeopsis masonii, Lilaeopsis occidentalis, Limnophyton, Ludwigia adscendens, Ludwigia palustris, Ludwigia peruviana, Luronium, Marsilea, Marsilea ancyclopoda, Marsilea crenata, Marsilea minuta, Marsilea quadrifolia, Monochoria korsakowii, Monochoria vaginalis, Najas, Najas ancistrocarpa, Najas filifolia, Najas flexilis, Najas gracillima, Najas graminea, Najas grossareolata, Najas guadalupensis, Najas hagerupii, Najas halophila, and Najas indica

In some aspects, species of aquatic plants may be selected from the following groups: Najas madagascariensis, Najas minor, Najas pseudogracillima, Najas tenuifolia, Najas tenuis, Najas wrightiana, Nuphar advena, Nuphar sagittifolia, Nuphar variegate, Nymphaeaceae, Phragmites australis, Pistia, Pogostemon stellatus, Pontederia, Pontederia cordata, Pontederiaceae, Potamogeton, Potamogetonaceae, Prionium, Rheophyte, Salvinia, Salvinia minima, Salvinia molesta, Salvinia natans, Sclerolepis, Sesbania, Shinnersia, Sparganium glomeratum, Spirodela, Stratiotes, Tagetes epapposa, Tagetes oaxacana, Tonina fluviatilis, Trichocoronis, Trithuria, Trithuria inconspicua, Typha, Typha capensis, Typha domingensis, Typha grossheimii, Typha laxmannii, Typha tzvelevii, Utricularia floridana, Utricularia olivacea, Vaccinium macrocarpon, Vesicularia montagnei, Victoria, Victoria amazonica, Victoria cruziana, Viguiera, Wolffiella gladiate, Xyris isoetifolia, and Zannichellia palustris

### II. Aquatic Plant and/or Algae Compositions

In some aspects, the aquatic plant and/or algae compositions comprise one or more plant and one or more algae.

In some aspects, the aquatic plant and/or algae compositions are prepared fresh, chopped, homogenized, blended, ground, powdered, or dried.

In some aspects, the aquatic plant and/or algae compositions include vitamins and/or metabolites thereof, minerals, trace elements, emulsifiers, aromatizing products, binders, colorants, odorants, thickening agents, antibiotics, and the like. In some aspects, the vitamins may be selected from vitamin B5, B1, B2, B3, B6, B9, B12, H, C, A, D, E, or K; and combinations thereof. In some aspects, the aquatic plant and/or algae compositions include microbes that synthesize vitamin B5, B1, B2, B3, B6, B9, B12, H, C, A, D, E, and/or K. In some aspects, the aquatic plant and/or algae compositions include microbes that synthesize vitamin B5. In some aspects, the metabolites of vitamin B5, B1, B2, B3, B6, B9, B12, H, C, A, D, E, or K are contemplated as one or more components of the aquatic plant and/or algae compositions.

In some aspects, the aquatic plant and/or algae compositions of the present disclosure comprise colorants including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. In some aspects, the aquatic plant and/or algae compositions of the present disclosure comprise trace nutrients such as salts of iron, manganese, boron, copper, cobalt, molybdenum and zinc. In some aspects, the aquatic plant and/or algae compositions comprise dyes, both natural and artificial.

In some aspects, the aquatic plant and/or algae compositions of the present disclosure comprise saccharides (e.g., monosaccharides, disaccharides, trisaccharides, polysaccharides, oligosaccharides, and the like), polymeric saccharides, lipids, polymeric lipids, lipopolysaccharides, proteins, polymeric proteins, lipoproteins, nucleic acids, nucleic acid polymers, silica, inorganic salts and combinations thereof.

In some aspects, the aquatic plant and/or algae compositions comprise polymers of agar, agarose, gelrite, gellan gum, and the like. In some aspects, the aquatic plant and/or algae compositions comprise plastic capsules, emulsions (e.g., water and oil), membranes, and artificial membranes. In some aspects, emulsions or linked polymer solutions may comprise aquatic plant and/or algae compositions of the present disclosure. See Harel and Bennett (U.S. Pat. No. 8,460,726 B2).

In some aspects, aquatic plant and/or algae compositions of the present disclosure comprise one or more oxygen scavengers, denitrifies, nitrifiers, heavy metal chelators, and/or dechlorinators; and combinations thereof. In one aspect, the one or more oxygen scavengers, denitrifiers, nitrifiers, heavy metal chelators, and/or dechlorinators are not chemically active once the aquatic plant and/or algae compositions are administered.

In some aspects, aquatic plant and/or algae compositions of the present disclosure occur in a solid form or a liquid form. In some aspects, aquatic plant and/or algae compositions of the present disclosure are added in dry form to a liquid to a liquid to form a suspension immediately prior to administration.

In some aspects, aquatic plant and/or algae compositions of the present disclosure comprise one or more preservatives. The preservatives may be in liquid or gas formulations. The preservatives may be selected from one or more of monosaccharide, disaccharide, trisaccharide, polysaccharide, acetic acid, ascorbic acid, calcium ascorbate, erythorbic acid, iso-ascorbic acid, erythrobic acid, potassium nitrate, sodium ascorbate, sodium erythorbate, sodium iso-ascorbate, sodium nitrate, sodium nitrite, nitrogen, benzoic acid, calcium sorbate, ethyl lauroyl arginate, methyl-p-hydroxy benzoate, methyl paraben, potassium acetate, potassium benzoiate, potassium bisulphite, potassium diacetate, potassium lactate, potassium metabisulphite, potassium sorbate, propyl-p-hydroxy benzoate, propyl paraben, sodium acetate, sodium benzoate, sodium bisulphite, sodium nitrite, sodium diacetate, sodium lactate, sodium metabisulphite, sodium salt of methyl-p-hydroxy benzoic acid, sodium salt of propyl-p-hydroxy benzoic acid, sodium sulphate, sodium sulfite, sodium dithionite, sulphurous acid, calcium propionate, dimethyl dicarbonate, natamycin, potassium sorbate, potassium bisulfite, potassium metabisulfite, propionic acid, sodium diacetate, sodium propionate, sodium sorbate, sorbic acid, ascorbic acid, ascorbyl palmitate, ascorbyl stearate, butylated hydro-xyanisole, butylated hydroxytoluene (BHT), butylated hydroxyl anisole (BHA), citric acid, citric acid esters of mono- and/or diglycerides, L-cysteine, L-cysteine hydrochloride, gum guaiacum, gum guaiac, lecithin, lecithin citrate, monoglyceride citrate, monoisopropyl citrate, propyl gallate, sodium metabisulphite, tartaric acid, tertiary butyl hydroquinone, stannous chloride, thiodipropionic acid, dilauryl thiodipropionate, distearyl thiodipropionate, ethoxyquin, sulfur dioxide, formic acid, or tocopherol(s).

In some aspects, the aquatic plant and/or algae compositions of the present disclosure possesses a water activity (aw) of less than 0.750, 0.700, 0.650, 0.600, 0.550, 0.500, 0.475, 0.450, 0.425, 0.400, 0.375, 0.350, 0.325, 0.300, 0.275, 0.250, 0.225, 0.200, 0.190, 0.180, 0.170, 0.160, 0.150, 0.140, 0.130, 0.120, 0.110, 0.100, 0.095, 0.090, 0.085, 0.080, 0.075, 0.070, 0.065, 0.060, 0.055, 0.050, 0.045, 0.040, 0.035, 0.030, 0.025, 0.020, 0.015, 0.010, or 0.005.

In some aspects, the aquatic plant and/or algae compositions of the present disclosure possesses a water activity (aw) of less than about 0.750, about 0.700, about 0.650, about 0.600, about 0.550, about 0.500, about 0.475, about 0.450, about 0.425, about 0.400, about 0.375, about 0.350, about 0.325, about 0.300, about 0.275, about 0.250, about 0.225, about 0.200, about 0.190, about 0.180, about 0.170, about 0.160, about 0.150, about 0.140, about 0.130, about 0.120, about 0.110, about 0.100, about 0.095, about 0.090, about 0.085, about 0.080, about 0.075, about 0.070, about 0.065, about 0.060, about 0.055, about 0.050, about 0.045, about 0.040, about 0.035, about 0.030, about 0.025, about 0.020, about 0.015, about 0.010, or about 0.005.

The water activity values are determined by the method of Saturated Aqueous Solutions (Multon, “Techniques d'Analyse E De Controle Dans Les Industries Agroalimentaires” APRIA (1981)) or by direct measurement using a viable Robotronic BT hygrometer or other hygrometer or hygroscope.

In some aspects, the aquatic plant and/or algae compositions, or a component thereof, is dried by desiccation, lyophilization, vitrification, foam drying, preservation by vaporization, vacuum-drying, or spray-drying.

In some aspects, the aquatic plant and/or algae compositions are formulated as a tablet, capsule, pill, food ingredient, food additive, food preparation, food supplement, consumable solution, consumable solid, consumable gel, injection, bolus, enema, suppository, or combinations thereof.

In some aspects, the aquatic plant and/or algae compositions is formulated as any type of edible product such as a powder, a bar, a smoothie, a yogurt, a shake, a syrup, etc.

In some aspects, the aquatic plant and/or algae compositions comprise one or more exogenous polyphenol. In some aspects, the aquatic plant and/or algae compositions comprise one or more exogenous phenolic acid, flavonoid, stilbene, and/or lignin is administered with the one or more aquatic plant or algae. In some aspects, the aquatic plant and/or algae compositions comprise one or more exogenous dietary fiber (soluble, insoluble, fermentable, or non-fermentable).

### III. Aquatic Plants and/or Algae Composition Administration

In some aspects, the aquatic plant and/or algae composition is administered, autologously, to a patient or subject in need via oral ingestion.

In some aspects, the aquatic plant and/or algae composition is administered in a dose volume comprising a total of, or at least, 0.1 ml, 0.2 ml, 0.3 ml, 0.4 ml, 0.5 ml, 0.6 ml, 0.7 ml, 0.8 ml, 0.9 ml, 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, 25 ml, 26 ml, 27 ml, 28 ml, 29 ml, 30 ml, 31 ml, 32 ml, 33 ml, 34 ml, 35 ml, 36 ml, 37 ml, 38 ml, 39 ml, 40 ml, 41 ml, 42 ml, 43 ml, 44 ml, 45 ml, 46 ml, 47 ml, 48 ml, 49 ml, 50 ml, 60 ml, 70 ml, 80 ml, 90 ml, 100 ml, 200 ml, 300 ml, 400 ml, 500 ml, 600 ml, 700 ml, 800 ml, 900 ml, or 1,000 ml.

In some aspects, the aquatic plant and/or algae composition is administered 1 or more times per day. In some aspects, the aquatic plant and/or algae composition is administered 1 or more times per week. In some aspects, the aquatic plant and/or algae composition is administered 1 or more times per month.

In some aspects, the aquatic plant and/or algae composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per day.

In some aspects, the aquatic plant and/or algae composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per week.

In some aspects, the aquatic plant and/or algae composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per month.

In some aspects, the aquatic plant and/or algae composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per year.

### IV. Aquatic Plants and/or Algae Administration Outcomes

In some aspects, the administration of the aquatic plant and/or algae composition to a subject yields a weight loss of least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, or 80% relative to a subject not having been administered the aquatic plant and/or algae composition.

In some aspects, the administration of the aquatic plant and/or algae composition to a subject yields a decrease of at least 20, 19, 18, 17, 16, 15, 14, 13, 12, 11, 10, 9, 8, 7, 6, 5, 4, 3, 2, or 1 BMI kg/m², relative to a subject not having been administered the aquatic plant and/or algae composition.

In some aspects, the compositions of the present disclosure are capable of shifting the microbiota of the gut in patients or subjects administered one or more of the compositions. In some aspects, the shift in the microbiome is an alteration of the microbial community such that the relative ratios of the microbes have shifted in patients or subjects that were administered one or more of the compositions, as compared to a sufficient experimental control in a person which has not been administered one or more of the compositions.

In some aspects, the shift of the microbiome is a shift in the relative ratios or the absolute abundance/activity of the following bacterial phyla: Firmicutes, Bacteroidetes, Actinobacteria, Verrucomicrobia, and Proteobacteria. In some aspects, the shift of the microbiome is a shift in the relative ratios or the absolute abundance of the following bacterial families: Ruminococcaceae, Lachnospiraceae, Bacteroidaceae, Veillonellaceae, Prevotellaceae, Bifidobacteriaceae, and Verrucomicrobiaceae.

In some aspects, administration of the aquatic plant and/or algae composition modulates the lipid and or glucose parameters profile measured by a standard serum lipid/glucose control parameters/glycemic profile. In further aspects, the modulation is an increase in the healthy parameters of the lipid/glycemic profile and a decrease in the unhealthy parameters of the lipid/glycemic profile. Administration of the aquatic plant and/or algae composition modulates profiles of any one or more of (1) lipids such as cholesterol (total, LDL and HDL-C), and triglycerides.

In some aspects, administration of the aquatic plant and/or algae composition results in an improvement of blood glucose and insulin regulation. In some aspects, administration of the aquatic plant and/or algae composition results in an improved oral glucose tolerance test (OGTT) as measured by blood glucose and insulin before, during, and at ˜120 minutes after ingestion of 75 g of glucose when compared to an initial OGTT or when compared to subjects receiving control (placebo) administrations. In some aspects, administration of the aquatic plant and/or algae composition results in an improvement in blood fasting glucose levels and insulin levels.

### Autologous Microbiome Reintroduction

In some aspects, the patient or subject is administered an aquatic plant and/or algae composition set forth in the present disclosure, and once an optimal sustained weight loss is attained—the subject, as a host, provides its own feces, which introduce and optimal state of its microbiome profile, including the gut microbiota diversity, genetic profile, functionality, viruses etc, reflecting the host optimal metabolic state. The idea is to retain the host his optimal gut microbiome state, captured in a good state, few months after, while the host is challenged with optional weight regain. The specific personal microbiome of the patient or subject is sampled and archived. In some aspects, the archived microbiome sample is later reintroduced to the same patient or subject from which it was collected as a means for attenuating or preventing the regain of the weight lost and/or improved lipid/glucose parameters while being administered the aquatic plant and/or algae composition and triggering its microbiome memory.

In some aspects, the patient or subject is not administered an aquatic plant and/or algae composition, and once an optimal sustained weight loss is attained from dieting and/or exercise, the microbiome of the patient or subject is sampled and archived. In some aspects, the archived microbiome sample is later reintroduced to the same patient or subject from which it was collected as a means for preventing a regain of the weight lost. In some aspects, the archived microbiome sample is later reintroduced to the same patient or subject , from which it was collected as a means for slowing wait regain, as compared to a patient or subject receiving a placebo control. In some aspects, a microbiome composition is administered to a patient or a subject to improve cardiometabolic health. In some aspects, a microbiome composition is administered to a patient or a subject to improve serum lipid/glucose profiles.

### I. Microbiome Sample Collection

In some aspects, the autologous gut microbiome sample may be collected from the feces, the mouth, the stomach, the small intestine, the large intestine, the duodenum, the jejunum, the ileum, the cecum, the ascending colon, the transverse colon, the descending colon, the sigmoid colon, the rectum, the anus, or a combination thereof from a patient or subject for the purpose of administering the sample to the same patient or subject at a point later in time.

In some aspects, the gut microbiome sample is collected from a person after an optimal/desired sustained weight loss as set forth herein.

### II. Microbiome Sample Processing & Storage

In some aspects, the collected microbiome sample is mixed with water, normal saline, or a buffered solution followed by a filtration step to filter out particulates, but to leave behind the microbes.

In some aspects, the resulting filtered sample is passaged on/in one or more types of solid or liquid microbial growth medias in order to achieve a higher number of colony forming units than what was collected from the patient or subject.

In some aspects, the resulting filtered sample is pooled together with other samples collected on the same day or same week.

In some aspects, the resulting filtered sample or the resulting passaged microbes are mixed with a sterile cryopreservative solution sufficient to mitigate freeze/thaw damage to microbes and maintained at a temperature between −180° C. and −20° C. In some aspects, the resulting filtered sample or the resulting passaged microbes are mixed with a sterile cryopreservative and frozen at a temperature between −180° C. and −20° and then administered to a subject or patient as a frozen capsule.

### III. Microbiome Formulation

In some aspects, the microbes of the microbiome sample are combined into microbiome compositions.

In some aspects, the microbiome compositions include vitamins and/or metabolites thereof, minerals, trace elements, emulsifiers, aromatizing products, binders, colorants, odorants, thickening agents, antibiotics, and the like.

In some aspects, the vitamins may be selected from vitamin B5, B1, B2, B3, B6, B9, B12, H, C, A, D, E, or K; and combinations thereof. In some aspects, the microbiome composition includes microbes that synthesize vitamin B5, B1, B2, B3, B6, B9, B12, H, C, A, D, E, and/or K. In some aspects, the microbiome compositions include microbes that synthesize vitamin B5. In some aspects, the metabolites of vitamin B5, B1, B2, B3, B6, B9, B12, H, C, A, D, E, or K are contemplated as one or more components of the microbiome composition.

In some aspects, the microbiome composition of the present disclosure comprises colorants including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. In some aspects, the microbiome compositions of the present disclosure comprise trace nutrients such as salts of iron, manganese, boron, copper, cobalt, molybdenum and zinc. In some aspects, the microbiome compositions comprise dyes, both natural and artificial.

In some aspects, microbiome compositions of the present disclosure comprise saccharides (e.g., monosaccharides, disaccharides, trisaccharides, polysaccharides, oligosaccharides, and the like), polymeric saccharides, lipids, polymeric lipids, lipopolysaccharides, proteins, polymeric proteins, lipoproteins, nucleic acids, nucleic acid polymers, silica, inorganic salts and combinations thereof.

In some aspects, microbiome compositions comprise polymers of agar, agarose, gelrite, gellan gum, and the like. In some aspects, microbiome compositions comprise plastic capsules, emulsions (e.g., water and oil), membranes, and artificial membranes. In some aspects, emulsions or linked polymer solutions may comprise microbiome compositions of the present disclosure. See Harel and Bennett (U.S. Pat. No. 8,460,726 B2).

In some aspects, microbiome compositions of the present disclosure comprise one or more oxygen scavengers, denitrifies, nitrifiers, heavy metal chelators, and/or dechlorinators; and combinations thereof. In one aspect, the one or more oxygen scavengers, denitrifiers, nitrifiers, heavy metal chelators, and/or dechlorinators are not chemically active once the microbiome compositions are administered.

In some aspects, microbiome compositions of the present disclosure occur in a solid form (e.g., dispersed lyophilized microbes) or a liquid form (microbes interspersed in a storage medium). In some aspects, microbiome compositions of the present disclosure are added in dry form to a liquid to a liquid to form a suspension immediately prior to administration

In some aspects, microbiome compositions of the present disclosure comprise one or more preservatives. The preservatives may be in liquid or gas formulations. The preservatives may be selected from one or more of monosaccharide, disaccharide, trisaccharide, polysaccharide, acetic acid, ascorbic acid, calcium ascorbate, erythorbic acid, iso-ascorbic acid, erythrobic acid, potassium nitrate, sodium ascorbate, sodium erythorbate, sodium iso-ascorbate, sodium nitrate, sodium nitrite, nitrogen, benzoic acid, calcium sorbate, ethyl lauroyl arginate, methyl-p-hydroxy benzoate, methyl paraben, potassium acetate, potassium benzoiate, potassium bisulphite, potassium diacetate, potassium lactate, potassium metabisulphite, potassium sorbate, propyl-p-hydroxy benzoate, propyl paraben, sodium acetate, sodium benzoate, sodium bisulphite, sodium nitrite, sodium diacetate, sodium lactate, sodium metabisulphite, sodium salt of methyl-p-hydroxy benzoic acid, sodium salt of propyl-p-hydroxy benzoic acid, sodium sulphate, sodium sulfite, sodium dithionite, sulphurous acid, calcium propionate, dimethyl dicarbonate, natamycin, potassium sorbate, potassium bisulfite, potassium metabisulfite, propionic acid, sodium diacetate, sodium propionate, sodium sorbate, sorbic acid, ascorbic acid, ascorbyl palmitate, ascorbyl stearate, butylated hydro-xyanisole, butylated hydroxytoluene (BHT), butylated hydroxyl anisole (BHA), citric acid, citric acid esters of mono- and/or diglycerides, L-cysteine, L-cysteine hydrochloride, gum guaiacum, gum guaiac, lecithin, lecithin citrate, monoglyceride citrate, monoisopropyl citrate, propyl gallate, sodium metabisulphite, tartaric acid, tertiary butyl hydroquinone, stannous chloride, thiodipropionic acid, dilauryl thiodipropionate, distearyl thiodipropionate, ethoxyquin, sulfur dioxide, formic acid, or tocopherol(s).

In some aspects, the microbiome compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some aspects, the microbiome compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some aspects, the microbiome compositions are shelf stable at room temperature (68¬72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some aspects, the microbiome compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some aspects, the microbiome compositions are shelf stable at -23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some aspects, the microbiome compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some aspects, the microbiome compositions are shelf stable at 77-100° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some aspects, the microbiome compositions are shelf stable at 77-100° F. fora period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some aspects, the microbiome compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some aspects, the microbiome compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some aspects, the microbiome compositions of the present disclosure are shelf stable at any of the disclosed temperatures and/or temperature ranges and spans of time at a relative humidity of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98%.

In some aspects, the microbiome compositions are shelf stable at −292 to −112° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some aspects, the microbiome compositions are shelf stable at −292 to −112° F. fora period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some aspects, the microbiome compositions of the present disclosure possesses a water activity (aw) of less than 0.750, 0.700, 0.650, 0.600, 0.550, 0.500, 0.475, 0.450, 0.425, 0.400, 0.375, 0.350, 0.325, 0.300, 0.275, 0.250, 0.225, 0.200, 0.190, 0.180, 0.170, 0.160, 0.150, 0.140, 0.130, 0.120, 0.110, 0.100, 0.095, 0.090, 0.085, 0.080, 0.075, 0.070, 0.065, 0.060, 0.055, 0.050, 0.045, 0.040, 0.035, 0.030, 0.025, 0.020, 0.015, 0.010, or 0.005.

In some aspects, the microbiome compositions of the present disclosure possesses a water activity (aw) of less than about 0.750, about 0.700, about 0.650, about 0.600, about 0.550, about 0.500, about 0.475, about 0.450, about 0.425, about 0.400, about 0.375, about 0.350, about 0.325, about 0.300, about 0.275, about 0.250, about 0.225, about 0.200, about 0.190, about 0.180, about 0.170, about 0.160, about 0.150, about 0.140, about 0.130, about 0.120, about 0.110, about 0.100, about 0.095, about 0.090, about 0.085, about 0.080, about 0.075, about 0.070, about 0.065, about 0.060, about 0.055, about 0.050, about 0.045, about 0.040, about 0.035, about 0.030, about 0.025, about 0.020, about 0.015, about 0.010, or about 0.005.

The water activity values are determined by the method of Saturated Aqueous Solutions (Multon, “Techniques d′Analyse E De Controle Dans Les Industries Agroalimentaires” APRIA (1981)) or by direct measurement using a viable Robotronic BT hygrometer or other hygrometer or hygroscope.

In some aspects, the microbiome composition, or a component thereof, is dried by desiccation, lyophilization, vitrification, foam drying, preservation by vaporization, vacuum-drying, or spray-drying.

In some aspects, the microbiome composition is formulated as a tablet, capsule, pill, food ingredient, food additive, food preparation, food supplement, consumable solution, beverage, consumable solid, consumable gel, injection, bolus, enema, suppository, or combinations thereof. In some aspects, the formulations are frozen and administered to the patient or subject frozen or first allowed to thaw.

In some aspects, the microbiome composition is formulated as any type of edible product such as a powder, a bar, a smoothie, a yogurt, a shake, a syrup, etc.

In some aspects, the microbiome composition comprises one or more exogenous polyphenol. In some aspects, the microbiome composition comprises one or more exogenous phenolic acid, flavonoid, stilbene, and/or lignin. In some aspects, the microbiome composition comprises one or more exogenous dietary fiber (soluble, insoluble, fermentable, or non-fermentable).

### IV. Microbiome Composition Administration

In some aspects, the microbiome composition is administered, autologously, to a patient or subject in need via oral ingestion, a nasogastric tube, nasojejunal tube, esophagogastroduodenoscopy, colonoscopy, retention enema, anally, or a combination thereof.

In some aspects, the microbiome composition is administered in a dose volume comprising a total of, or at least, 0.1 ml, 0.2 ml, 0.3 ml, 0.4 ml, 0.5 ml, 0.6 ml, 0.7 ml, 0.8 ml, 0.9 ml, 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, 25 ml, 26 ml, 27 ml, 28 ml, 29 ml, 30 ml, 31 ml, 32 ml, 33 ml, 34 ml, 35 ml, 36 ml, 37 ml, 38 ml, 39 ml, 40 ml, 41 ml, 42 ml, 43 ml, 44 ml, 45 ml, 46 ml, 47 ml, 48 ml, 49 ml, 50 ml, 60 ml, 70 ml, 80 ml, 90 ml, 100 ml, 200 ml, 300 ml, 400 ml, 500 ml, 600 ml, 700 ml, 800 ml, 900 ml, or 1,000 ml.

In some aspects, the microbiome composition is administered in a dose comprising a total of, or at least, 10¹⁸, 10¹⁷, 10¹⁶, 10¹⁵, 10¹⁴, 10¹³, 10¹², 10¹¹, 10¹⁰, 10⁹, 10⁸, 10⁷, 10⁶, 10⁵, 10⁴, 10³, or 10² microbial cells.

In some aspects, the administered dose of the microbiome composition comprises 102 to 10¹⁸, 10³ to 10¹⁸, 10⁴ to 10¹⁸, 10⁵ to 10¹⁸, 10⁶ to 10¹⁸, 10⁷ to 10¹⁸, 10⁸ to 10¹⁸, 10⁹ to 10¹⁸, 10¹⁰ to 10¹⁸, 10¹¹ to 10¹⁸, 10¹² to 10¹⁸, 10¹³ to 10¹⁸, 10¹⁴ to 10¹⁸, 10¹⁵ to 10¹⁸, 10¹⁶ to 10¹⁸, 10¹⁷ to 10¹⁸, 10² to 10¹², 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 1011, 10⁸ to 10¹¹, 10⁹ to 10¹¹, 10¹⁰ to 10¹¹, 10² to 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 109, 10⁵ to 10⁹, 10⁶ to 10⁹, 10⁷ to 10⁹, 10⁸ to 10⁹, 10² to 10⁸, 10³ to 10⁸, 10⁴ to 10⁸, 10⁵ to 10⁸, 10⁶ to 10⁸, 10⁷ to 10⁸, 10² to 10⁷, 10³ to 10⁷, 10⁴ to 10⁷, 10⁵ to 10⁷, 10⁶ to 10⁷, 10² to 10⁶, 10³ to 10⁶, 10⁴ to 10⁶, 10⁵ to 10⁶, 10² to 10⁵, 10³ to 10⁵, 10⁴ to 10⁵, 10² to 10⁴, 10³ to 10⁴, 10² to 10³, 10¹⁸, 10¹⁷, 10¹⁶, 10¹⁵, 10¹⁴, 10¹³, 10¹², 10¹¹, 10¹⁰, 10⁹, 10⁸, 10⁷, 10⁶, 10⁵, 10⁴, 10³, or 10² total microbial cells.

In some aspects, the microbiome composition is administered 1 or more times per day. In some aspects, the microbiome composition is administered 1 or more times per week. In some aspects, the microbiome composition is administered 1 or more times per month.

In some aspects, the microbiome composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per week.

In some aspects, the microbiome composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per month.

In some aspects, the microbiome composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per year.

In some aspects, the microbes or microbiome composition is administered in a time-released fashion. The composition may be coated in a chemical composition, or the composition may be contained in a mechanical device or capsule that releases the microbes or microbiome composition over a period of time instead all at once. In one aspect, the microbes or microbiome composition is administered in a time-release capsule. In one aspect, the composition may be coated in a chemical composition, or may be contained in a mechanical device or capsule that releases the microbes or microbiome compositions all at once a period of time hours post-administration.

In some aspects, the microbes or microbiome composition is released post-administered in a time-released fashion between 1 to 5, 1 to 10, 1 to 15, 1 to 20, 1 to 24, 1 to 25, 1 to 30, 1 to 35, 1 to 40, 1 to 45, 1 to 50, 1 to 55, 1 to 60, 1 to 65, 1 to 70, 1 to 75, 1 to 80, 1 to 85, 1 to 90, 1 to 95, or 1 to 100 hours.

In some aspects, the microbes or microbiome composition is released post-administration in a time-released fashion between 1 to 2, 1 to 3, 1 to 4, 1 to 5, 1 to 6, 1 to 7, 1 to 8, 1 to 9, 1 to 10, to 11, 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 29, or 1 to 30 days.

### V. Microbiome Composition Administration Outcomes

In some aspects, the administration of the microbiome composition to a subject yields a weight loss of least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, or 80% relative to a subject not having been administered the microbiome composition.

In some aspects, the administration of the microbiome composition to a subject yields a decrease of at least 20, 19, 18, 17, 16, 15, 14, 13, 12, 11, 10, 9, 8, 7, 6, 5, 4, 3, 2, or 1 BMI kg/m², relative to a subject not having been administered the microbiome composition.

In some aspects, the compositions of the present disclosure are capable of shifting the microbiota of the gut in patients or subjects administered one or more of the compositions. In some aspects, the shift in the microbiome is an alteration of the microbial community such that the relative ratios of the microbes have shifted in patients or subjects that were administered one or more of the compositions, as compared to a sufficient experimental control in a person which has not been administered one or more of the compositions.

In some aspects, the shift of the microbiome is a shift in the relative ratios or the absolute abundance of the following bacterial phyla such as: Firmicutes, Bacteroidetes, Actinobacteria, Verrucomicrobia, and Proteobacteria. In some aspects, the shift of the microbiome is a shift in the relative ratios or the absolute abundance of the following bacterial families such as: Ruminococcaceae, Lachnospiraceae, Bacteroidaceae, Veillonellaceae, Prevotellaceae, Bifidobacteriaceae, and Verrucomicrobiaceae.

In some aspects, the patient receiving the autologous microbiome composition maintains their weight for the duration in which the microbiome composition is being administered. In some aspects, the patient receiving the autologous microbiome composition maintains their weight for at least 1, 2, 3, 4, 5, 6, 7, 8, 9, 10, 11, or 12 months after the final administration of the microbiome composition.

In some aspects, administration of the microbiome composition modulates the lipid/glucose profile measured by a standard serum lipid/glucose profile. In further aspects, the modulation is an increase in the healthy parameters of the lipid/glucose profile and a decrease in the unhealthy parameters of the lipid profile. Administration of the microbiome composition modulates profiles of any one or more of (1) lipids such as cholesterol (total and HDL-C), and triglycerides; and (2) lipoproteins such as chylomicrons, very low-density lipoprotein (VLDL, intermediate-density lipoprotein (IDL), low-density lipoprotein (LDL), and high-density lipoprotein (HDL).

In some aspects, administration of the microbiome composition results in an improvement of blood glucose regulation. In some aspects, administration of the microbiome composition results in an improved oral glucose tolerance test (OGTT) as measured by blood glucose and insulin before, during, and at ˜120 minutes after ingestion of 75 g of glucose when compared to an initial OGTT or when compared to subjects receiving control (placebo) administrations. In some aspects, administration of the microbiome composition results in an improvement in blood fasting glucose levels and insulin levels.

### EXAMPLES

### Example 1A˜Identification of endophyte populations associated with Wolffia globosa, Mankai Varietal

Experiments were conducted to identify endophytes associated with Mankai that might play a major role in plant growth and in enriching its nutritional composition. Mankai was grown under sterile conditions, and the bacteria population obtained after crushing the plant was examined to identify the bacteria. Some of the identified bacteria isolated from a sterile culture of Mankai are listed below in Table 1.

TABLE 1
Bacteria isolated from a sterile culture of Wolffia globosa, Mankai varietal

| | | | | Rant Gowth | |
|---|---|---|---|---|---|
| | | Association with | | Promoting | Additional |
| no. | Bacteria ID | plant/medium | Host | bacteria (PGPB) | information |
| 14 | Methylobacterium Radiotolerans | medium/on plant | | | |
| 46 | Bosea lupini/robiniae [symbol] | unknown | Plant, Soil | | |
| | [symbol] | | | | |
| 47 | Chryseobacterium sp | unknown | Plant, animal, fish Soil | yes | B12 transport system |
| 48 | Spphingomonasaquatilis | unknown | duckweed? | yes | IAA production, |
| | | | | | ACCdeaminase |
| 49 | Rectobacillus roseus | unknown | aquatic enviroment | | |
| 51 | Cryptococcuslaurentii-yeast | unknown | plant area | | |
| 52 | Microbacterium arthrosphaerae | endophyte | plant (tomato), | yes | IAA production, |
| | | | nematode | | Nitrogen fixation |
| 53 | Bacillus niacini | endophyte | plant endophyte | yes | |
| | | | (soybean, green onion) | | |
| 55 | Hymenobacter gelipurpurascens | endophyte | soil | | |
| 56 | Arthrobacter agilis | endophyte | plant (azolla) | yes | iron uptake, Nitrogen |
| | | | | | fixation, known |
| | | | | | interaction with azolla |
| 57 | Moraxella_sg_/Moraxella osloensis | endophyte | plant (symbiosis | | |
| | | | with nematode) | | |
| 58 | Terri bacillusaidingensis/ | endophyte | | | |
| | saccharophilus/Virgibadiluspicturae | | | | |
| | [symbol] | | | | |
| 59 | Pantoea septica | endophyte | Can be found in | | |
| | | | human stool | | |

[symbol]

indicates data missing or illegible when filed

### Example 1B —Identification of Endophyte Populations Associated with Wolffia globosa, Mankai Varietal

Methods: To thoroughly investigate endophytic bacterial communities and to potentially exploit their beneficial effects by means of inoculation, the organisms must be cultivable under laboratory conditions. Organism isolation is the first critical step in this cultivation procedure. A common endophyte isolation protocol consists of a surface sterilization of the plant tissues followed by crushing the plant tissues and plating the slurry onto nutrient medium. Mankai™ culture was tested for endophyte population by disinfecting with Hydrogen peroxide treatment and the procedure was done as follows: Mankai™ was harvested from an outdoor cultivation basin, washed for 2 min with tap water and vacuum dried for 1 min. The plant (25 g) was then transferred to 500 ml plastic bottle with 125 ml of the various H₂O₂ solutions (0-7%) and placed in a laboratory rotator for 5 min. The solution was discarded and plant was washed twice with 125 ml of ultra-filtrated water for 1 min while rotating gently. About 30 mg of plant were plated on PCA agar plates (whole plants). Additional 30 mg were transferred to a sterile 1.5 ml tube, crushed with plastic pestle in 100 μl phosphate buffer and 30 μl were plated on a petri dish with PCA agar. All plates were incubated for 72 h at 30° C.

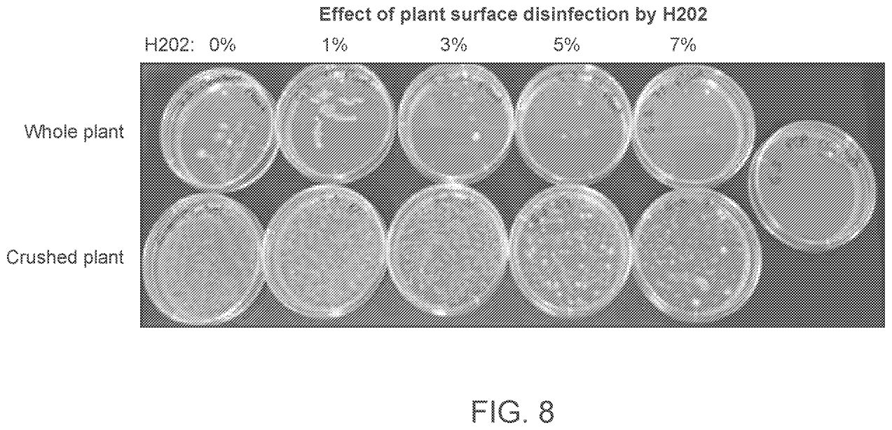

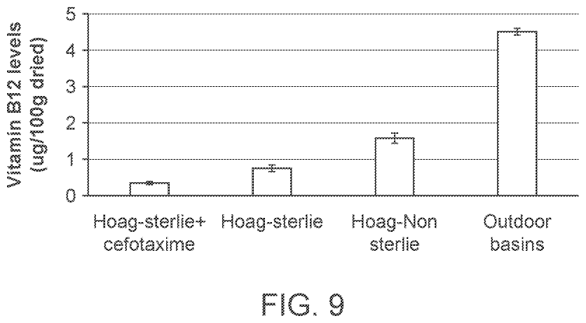

Results: The results showed that although plant surface was disinfected, many bacteria were observed on the PCA plate (total aerobic bacteria) after crushing the plant, suggesting a close association between the plant and the bacteria that may indicate a plant-endophyte relationship (FIG. 8).

Several potential endophytes were isolated from the H2O2 surface disinfected crushed plants. Selected clones were isolated and identified by MALDI or by 16S sequencing at Hy Laboratories Ltd (Israel). An indication on the potential contribution of those endophytes to the plant obtained from the literature is summarized in Table 2.

TABLE 2
Mankai isolated clones Bacteria endophytes ID—exampled list

| | Hinoman isolated | | |
|---|---|---|---|
| | clones | | |
| no. | Bacteria ID | Host/Habitat | Additional information |
| 1 | Methylobacterium | Endophytes | pink-pigmented |
| | Radiotolerans | and epiphytes | facultative |
| | | of various | methylotrophic (PPFM) |
| | | plants | bacteria, which |
| | | Habitat: plant, | are able to synthesize |
| | | soil, water, | carotenoids |
| | | sediment | |
| 2 | Bosea lupini / | Root nodules | Bacteria associate with |
| | robiniae *A | endophyte | legume |
| | single | Host: Lupinus | |
| | identification | polyphyllus, | |
| | could not be | Lathyrus | |
| | obtained. | latifolius, | |
| | | Robinia | |
| | | pseudoacacia | |
| 3 | Chryseobacterium | Can be found | Assosiation with B12 |
| | sp | in a wide | transport system |
| | | variety of | |
| | | environments. | |
| 4 | Sphingomonas | Various | Aerobic, yellow pigment |
| | aquatilis | species were | □producing bacterium. |
| | | found on 26 | Producing sphingolipids |
| | | plant species | & IAA |
| | | belonging to | |
| | | 11 families. | |
| 5 | Microbacterium | Members of | Gram-positive, |
| | arthrosphaerae | the genus can | non-spore-forming, |
| | | be isolated | rod-shaped |
| | | from a wide | bacteria. |
| | | range of | IAA production. |
| | | different | Nitrogen fixation |
| | | environmental | |
| | | habitats from | |
| | | soil to insects | |
| | | to human | |
| | | clinical | |
| | | specimens to | |
| | | marine | |
| | | environments | |
| | | and plants | |
| 6 | Bacillus niacini | Plant growth | Enhance plant growth by: |
| | | promoting | 1) mobilising phosphorus, |
| | | bacteria | 2) producing indoles, NH3 |
| | | | and proteases, as well as |
| | | | 3) conferring resistance to |
| | | | heavy metals |
| 7 | Arthrobacter | Plant growth | Produces amino lipid. |
| | agilis | promoting | Induces iron acquisition |
| | | rhizobacteria | Produce volatile organic |
| | | | compounds |

### Example 1C: Mankai Endophytes Exert Significant Effect on Plant Composition

A study of the potential contribution microbiome to the plant composition was performed using plant cultures under different microbial levels. As an example for compositional change we determined levels of B12 vitamin in the plant.

Methods: The experiment was performed with the following treatments: 1. Sterilized Mankai™ grown in the lab in Hoagland medium. 2. Sterilized Mankai™ grown in the lab in Hoagland medium and with supplementation of antibiotics cefotaxime. 3. Mankai™ from outside culture that was further grown indoors in Hoagland medium. 4. Mankai™ grown outdoor condition (Green-house). In the first two treatments-sterilization has been achieved by submersing the plant tissues into a predetermined concentration of sodium hypochlorite for 1-5 min. Afterwards, the plant tissues were washed several times in sterilized distilled water and plated onto sterile Hoagland solution supplemented with 0.5% sucrose to verify sterility. Post 1-2 days, bleached mother fronds with green daughter fronds were transferred to new vessels. In the following days the mother fronds died and the daughter fronds grew to establish sterilized Mankai™ culture. The sterilized plant was grown in Hoagland solution from about □250 mg to □25grams, with or without cefotaxime (treatment 2 and 1 respectively) in 15 cm petri dishes at 25° C., 24 h light photo period and light intensity of 120 μE. Medium was replaced every 5-7 days to maintain pH levels. Post two weeks, plant was harvested, washed and then dried for 16 h at 65° C. Outdoor plant that was transferred into the lab (treatment 3) grew in a small tank with circulation pump with similar temperature and light conditions as described above. Outdoor plant of treatment 4 was taken directly from cultivation basins and dried as described. All treatments were done in 5 independent replicates and one gram of each dried sample was used to determine B12 levels by using the Vitafast kit (r-biopharm, Germany), performed according to the manufacturer's instructions.

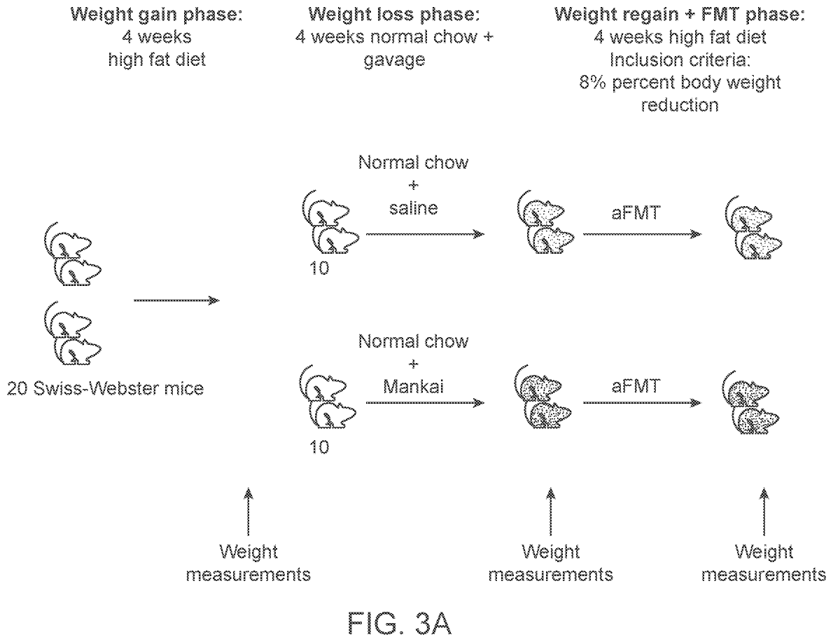

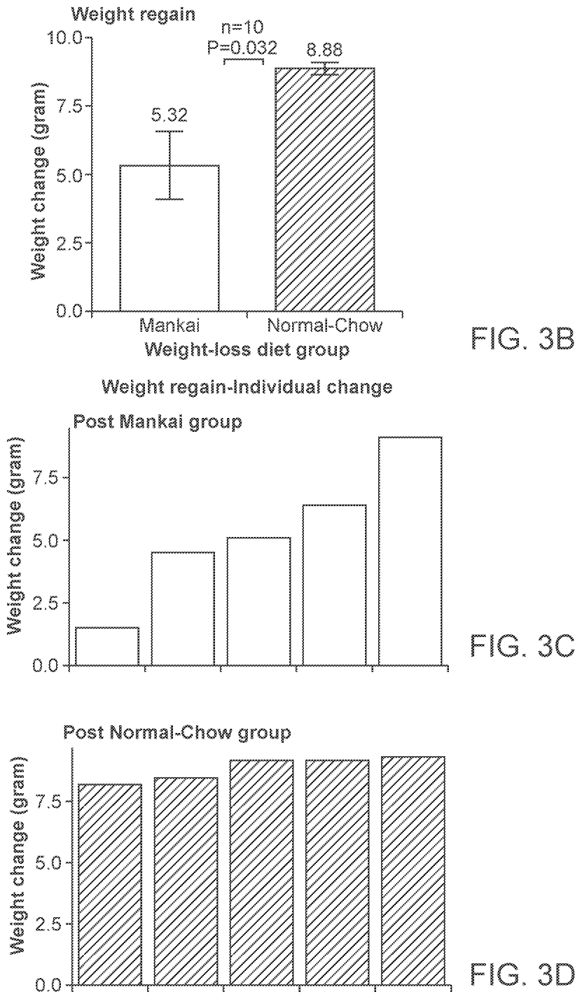

Results: The results clearly revealed that B12 vitamin Mankai levels are reduced in correlation with the reduction in microbial load (FIG. 3).

### Example 1D: Mankai Endophytes Exert Symbiotic Interaction with the Plant and its Environment

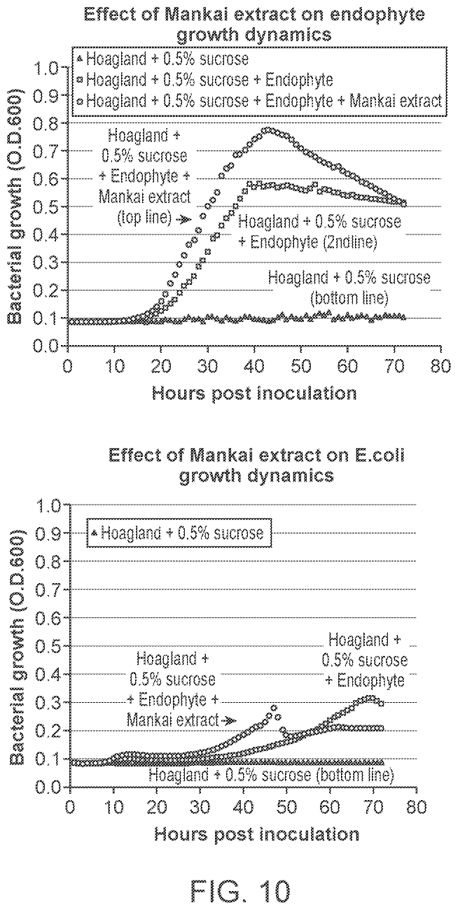

A. Methods: We compared the dynamic growth of an isolated Mankai endophyte bacteria vs. other bacteria (E. coli) in Hoaglant medium in different growth mediums (3 repetitions in each medium) as detailed in FIG. 10.

A. Results: The isolated Mankai endophyte bacteria grows superiorly with the Mankai plant extraction while the growth of the other bacteria (E. coli) is unaffected. This finding reinforces the assumption that the bacteria is indeed an endophyte of the Mankai plant and that the plant intrinsic microbiome interact with the plant environment. It is likely that the plant intrinsic microbiome receives from the plant a carbon or sugar source but it is unknown what the plant receives from the bacterium. The results suggest a potency of symbiotic environmental bacterial interaction.

B. Methods. To explore whether Mankai affects its water environment microbiome—Mankai™ outdoor cultivation basin was cleaned with 0.5% sodium hypochlorite solution and washed, and then treated with Steam high pressure cleaning system. Basin was filled with cultivation medium and the microbial load dynamics of different types of bacterial populations was measured by pour plate method in accordance with Israeli standard regulations (885.3).

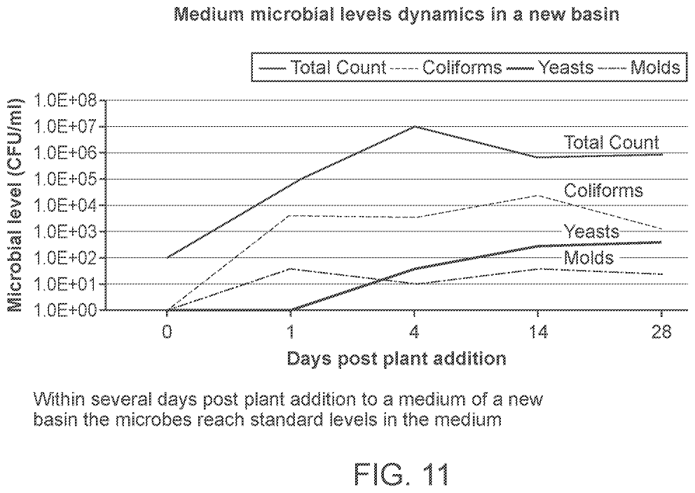

B. Results: The results show (FIG. 11) that on time 0, before the addition of the plant, microbial levels were low. After the addition of the plant an increase in the microbial load was observed and post 4 days the microbes reach the standard levels that are usually found in the medium during cultivation. The results suggest that the Mankai plant is potent in its effect on its extremal microbiome environment. Mankai endophytes probably exert symbiotic interaction with the plant and its environment.

### Example 2—Effect of Mankai-Induced Fecal Transplantation on Obesity and Glucose Metabolism in Mice Model of Obesity

In a 12-week experiment, 20 mice with obesity (achieved by a 4-week high-fat diet) were divided to 2 groups undergoing a 4-week weight-loss period by a normal-chow diet, with the supplementation of: (group 1) Mankai; (group 2) Normal-saline; by a daily gavage. Mankai dosage was 20 mg/g dry Mankai per day. Fecal samples were collected following the weight-loss phase and stored for later aFMT administration. In the final phase, the successful weight losers underwent a second 4-week weight gain (weight regain cycle) by high-fat diet, with the supplementation of weekly weight-nadir-based aFMT gavage. See FIG. 3A. Weight was assessed at baseline and following each weight loss/gain phase. Furthermore, intraperitoneal glucose tolerance test was performed to compare the clearance of an intraperitoneally injected glucose load of Mankai to control aFMT mice.

Out of the initial group, 10 mice that lost >8% body weight (mean=−10.5%) continued to the second phase. Following a 4-week high-fat diet phase, the Mankai enriched group achieved a significant halt of weight regain, as compared to the control group (ΔMankai=+5.32 gr (11.3% regain) vs. ΔControl+8.88 gr (17.6% regain); Pv=0.03). See FIGS. 3B-3D. Both groups of mice were administered aFMT, however, only mice that were administered Mankai showed a significant attenuation of weight regain, suggesting that the addition of Mankai to the diet resulted in alteration of the microbiome and thereby, attenuation of weight regain, following weight loss.

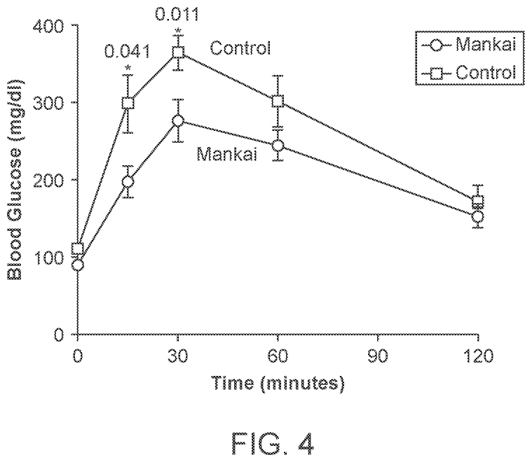

The intraperitoneal glucose tolerance test revealed that within 2 hours the Mankai aFMT mice curve was always lower than the control aFMT mice curve, with significant differences in the points related to the peak glucose levels (p<0.05). See FIG. 4. In obese mice, Mankai supplementation during weight-loss induces a more potent fecal microbiome, as to its ability to maintain subsequent body weight loss and a better glycemic response to glucose load in the regain phase. Mice that were administered Mankai showed better glycemic response to glucose load, as compared to mice on regular diet, suggesting that Mankai may be effective in regulating glucose metabolism and obesity. Furthermore, these data suggest that Mankai may be effective in altering/optimizing the internal microbiota to bring about a more favorable microbiota state, which attenuates the rebound of weight loss associated parameters (such as fasting or post prandial/load plasma insulin and fasting or post prandial/load plasma glucose) after a period of weight loss.

### Example 3 Effect of Administering Mankai on Humans

Methods: A two-phase randomized controlled trial involving overweight sedentary adults was performed in the DIRECT PLUS (ClinicalTrials.gov Identifier: NCT03020186) weight-loss trial. The trial included two phases: (1) a randomized, open-label, lifestyle intervention (2) a randomized, double-blind, autologous fecal microbiota transplantation (aFMT) augmentation. In the first phase, participants were randomly assigned at a 1:1:1 ratio to one of three lifestyle intervention groups: physical activity (PA); PA+Mediterranean (MED) diet or PA+Green-MED diet, as detailed in Table 3. Following 6-months of lifestyle intervention, participants who lost at least 3.5% body-weight were recruited to the double blind, placebo controlled, aFMT intervention. The 3.5% cut-off was chosen by an estimation aimed to maximize statistical power, accounting for both expected sample size and weight difference.

TABLE 3
Outline of dietary and PA interventions

| | PA + healthy | | |
|---|---|---|---|
| | dietary guidance | PA + MED | PA + green-MED |
| Physical activity | 18-months free gym membership, | | |
| | 18-months PA education sessions | | |
| | 45-60 minutes of aerobic training + resistance training, 3-4 times/week. | | |
| Lifestyle group sessions | 18-months group sessions in the workplace, weekly for the first month, and monthly | | |
| | thereafter | | |
| General dietary guidance | Limit dietary cholesterol, trans-fat, saturated-fat, sugars, and salt and increase intake | | |
| | of vegetables | | |
| Energy, kcal/day | Guidelines for a | 1500-1800 kcal/day for men, 1200-1400 kca1/day | |
| | healthy | for women | |
| Total fat, % of daily | MED diet with no | ~40% mainly PUFA | |
| consumption | specific recipes or | and MUFA | |
| Carbohydrates, gr/day | calorie restriction | Less than 40 gr/day in the first 2 months with increased | |
| | | gradual intake up to 80 gr/day | |
| Specific recommendations | | Avoid red and processed meats. Reduced poultry intake. | |
| Polyphenols, mg/day | | +440 mg/day | +1240 mg/day |
| | | [source: provided | [source: provided |
| | | walnuts (28 g/day] | walnuts (28 g/day), |
| | | | green tea |
| | | | (4 cups/day), Wolffia |
| | | | globosa duckweed (Mankai) |
| | | | shake (100 g frozen cubes)] |
| Specific recommendations | | | |

Eligible participants were asked to deliver a full fecal sample that was processed into aFMT capsules. The participants were simultaneously randomized, in a 1:1 ratio, to receive 10 grams of aFMT (consumed as ten 1 gram capsules) or identical placebo capsules delivered 10 times starting at 8-months following initiation of lifestyle intervention (2 months following fecal collection), over a 6-month period. Thus, a total of ˜100 g fecal matter was consumed between months 8 and 14. In order to robustly, and more persistently, counteract weight regain, the protocol was designed to comprise a 6-month administration period (months 8-14).

Participants were weighed without shoes to the nearest 0.1 kg at each administration session and at baseline, 6 and 14-months. Waist circumference (WC) was measured halfway between the last rib and the iliac crest to the nearest millimeter at baseline, 6, and 14-months. An online symptoms questionnaire, based on Common Terminology Criteria for Adverse Events version 5.0, available at www.meddra.org, was used to assess possible adverse effects 24-h after intake of capsules, following the 1^{st}, 4^{th}, 6^{th} and 8^{th} administration session. Blood samples were obtained after a 12-hour fast at baseline, 6 and 14 months, centrifuged and stored at 80° C. until assayed. Plasma insulin levels were measured with the use of an enzymatic immunometric assay [Immulite automated analyzer, Diagnostic Products, coefficient of variation (CV)=2.5%].

Fecal samples were collected at baseline, 6 and 14 months at the study site, immediately frozen to −20° C. for 1-3 days, then transferred to −80° C. pending DNA extraction. To characterize the microbiome, fecal DNA was extracted, sequenced and normalized with an average depth of 15.4±2.6 million reads per sample (mean±standard deviation). DNA sequences were aligned using an accelerated version of the Needleman-Wunsch algorithm to a curated database containing all representative genomes in RefSeq v86. Each input sequence was assigned the lowest common ancestor that was consistent across at least 80% of all reference sequences tied for best hit. The number of counts for each taxon was then normalized to the average genome length. Species accounting for less than one millionth of all species-level markers were discarded. Fecal samples from the 0 and 6-month time points were sequenced on a MiSeq platform following amplification of V3-V4 hypervariable region of the 16S rRNA gene using the primer set 341F/806R, and processed by the DADA2 pipeline, as described in Callahan B J, Mcmurdie P J, Rosen M J, Han A W, Johnson A J A, Holmes S P. dada2: high-resolution sample inference from illumina amplicon data. 2016; 13(7).

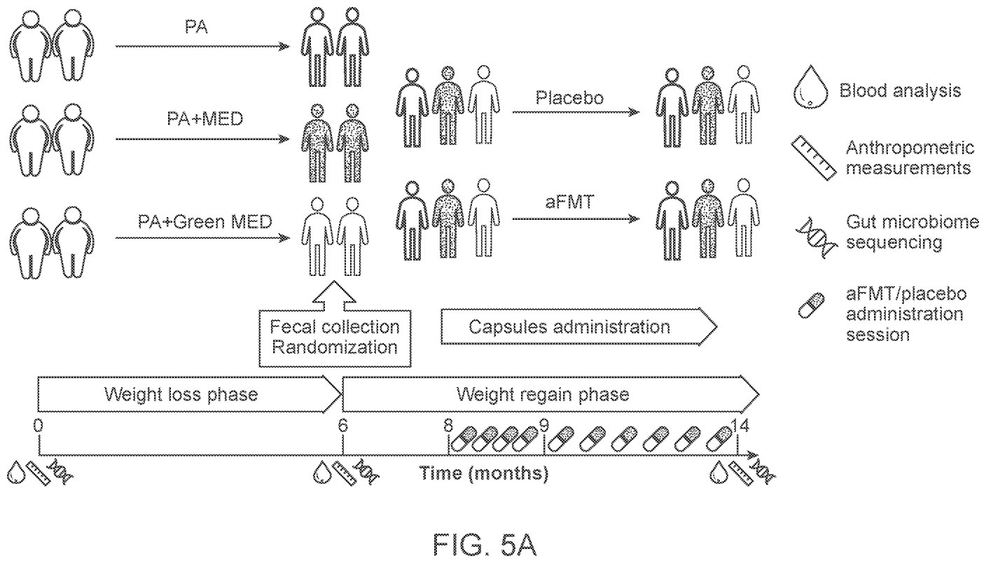

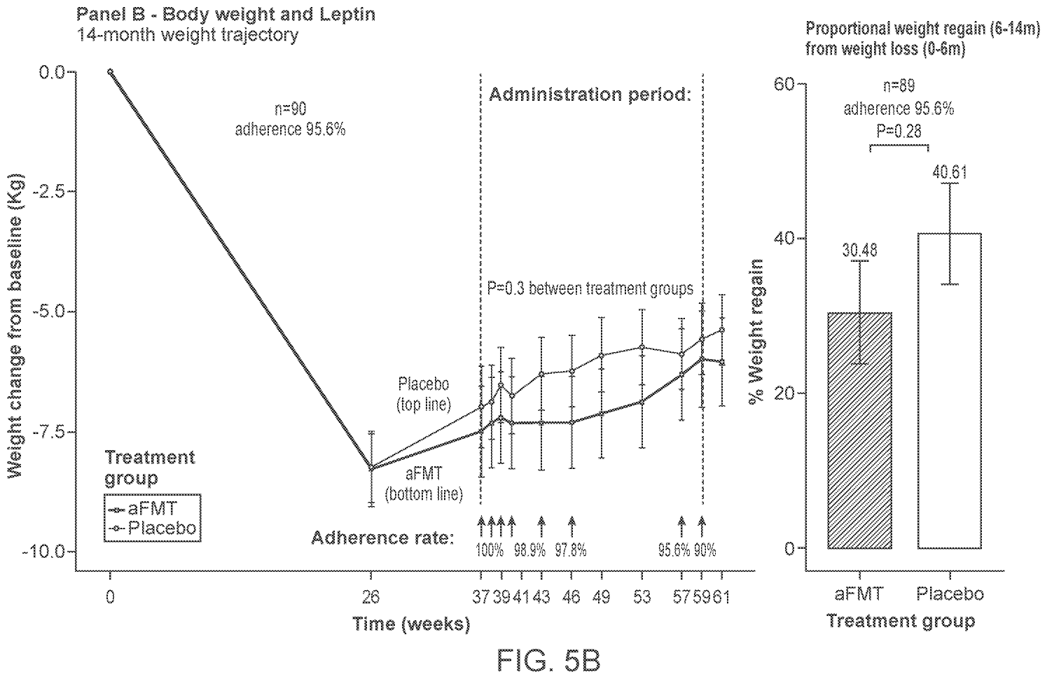

Results: Following 6 months of dietary intervention, 155 of 294 (52.7%) DIRECT-PLUS participants met the inclusion criteria of >3.5% 6-month weight-loss with no recent antibiotic therapy. Of these, 90 subjects who consented were randomly assigned to aFMT (n=44) or placebo (n=46) (FIG. 5). No significant differences in weight, anthropometric or metabolic characteristics were observed at 6-months and in 0-6 months changes between the 90 enrolled subjects to the 65 who declined.

Baseline and 6-month characteristics of the participants, across treatment and lifestyle intervention groups, are presented in Table 4. At baseline, mean age was 52 years and mean body mass index (BMI) was 31.3 kg/m. 91% of participants were male. Following 6-months of lifestyle intervention, mean weight-loss was −8.27±5 Kg. No significant differences were observed between the randomized treatment groups in anthropometric or laboratory measures at 6-months, in total or within lifestyle intervention group strata. The overall treatment compliance rate, defined as intake of >80 capsules, was 95.6%.

TABLE 4
Baseline characteristics of the study population

| | | | | | | | PA+ Green | |
|---|---|---|---|---|---|---|---|---|
| | All subjects | | PA group | | PA+ MED | | MED group | |
| Treatment | aFMT | Placebo | aFMT | Placebo | aFMT | Placebo | aFMT | Placebo |
| Subjects - no. | 44 | 46 | 8 | 8 | 17 | 18 | 19 | 20 |
| Male gender - no. | 42 | 40 | 7 | 8 | 16 | 15 | 19 | 17 |
| Age - yr (sd) | 53.14 | 51.63 | 52.43 | 52.05 | 54.49 | 52.61 | 52.24 | 50.57 |
| | (9.97) | (11.65) | (7.55) | (12.17) | (9.88) | (10.75) | (11.20) | (12.71) |
| Baseline Body | 30.89 | 31.39 | 30.57 | 29.66 | 30.93 | 31.92 | 30.97 | 31.60 |
| mass index (sd) | (3.45) | (4.09) | (3.81) | (2.11) | (3.77) | (5.28) | (3.19) | (3.40) |
| Baseline Waist | 108.91 | 109.59 | 108.25 | 102.38 | 109.18 | 113.17 | 108.95 | 109.25 |
| circumference- | (7.44) | (10.55) | (7.63) | (5.37) | (7.44) | (13.72) | (7.76) | (7.12) |
| cm (sd) | | | | | | | | |
| Baseline Weight- | 93.74 | 92.20 | 90.41 | 83.22 | 93.12 | 96.24 | 95.70 | 92.16 |
| kg (sd) | (14.12) | (14.45) | (15.44) | (9.82) | (13.58) | (17.24) | (14.50) | (11.98) |
| 6-months weight- | −8.28 | −8.25 | −6.20 | −4.65 | −8.23 | −9.38 | −9.21 | −8.68 |
| loss - kg (sd) | (5.16) | (4.85) | (3.06) | (2.26) | (4.01) | (5.27) | (6.54) | (4.71) |

6-months characteristics

| | | | | | | | | |
|---|---|---|---|---|---|---|---|---|
| Weight - kg (sd) | 85.46 | 83.95 | 84.21 | 78.58 | 84.89 | 86.86 | 86.49 | 83.48 |
| | (12.30) | (11.74) | (14.62) | (8.25) | (12.54) | (12.87) | (11.67) | (11.52) |
| Waist | 99.30 | 99.22 | 98.38 | 97.75 | 99.76 | 101.89 | 99.26 | 97.40 |
| circumference- | (7.32) | (8.02) | (8.25) | (6.80) | (8.50) | (6.94) | (6.06) | (9.02) |
| cm (sd) | | | | | | | | |
| Fasting or post | 97.12 | 94.71 | 93.39 | 92.17 | 94.63 | 94.17 | 101.14 | 96.22 |
| prandial/load | (14.78) | (8.30) | (19.90) | (6.72) | (7.18) | (8.07) | (17.26) | (9.10) |
| plasma glucose- | | | | | | | | |
| mg/dl (sd) | | | | | | | | |
| Fasting or post | 10.17 | 10.63 | 9.38 | 11.99 | 9.18 | 10.09 | 11.54 | 10.57 |
| prandial/load | (5.08) | (5.12) | (5.54) | (5.36) | (3.54) | (3.50) | (6.06) | (6.28) |
| plasma insulin- | | | | | | | | |
| microU/ml (sd) | | | | | | | | |

Values are presented as means (standard deviation). No significant differences were observed between placebo or aFMT group in the measured baseline characteristics, overall and across lifestyle interventions.

No severe adverse events were reported during the study period. The aFMT and placebo groups experienced a similar 0 to 6-month (i.e., baseline) weight-loss (aFMT=−8.28Kg vs. placebo=−8.25 Kg, P=0.91). The 6-14m weight change was +2.2kg (30.4% proportional weight regain from 0-6 m) in the aFMT group vs. +2.6kg (40.6% proportional regain) in the placebo (P=0.3 for weight change, P=0.28 for proportional weight regain) (FIG. 5B).

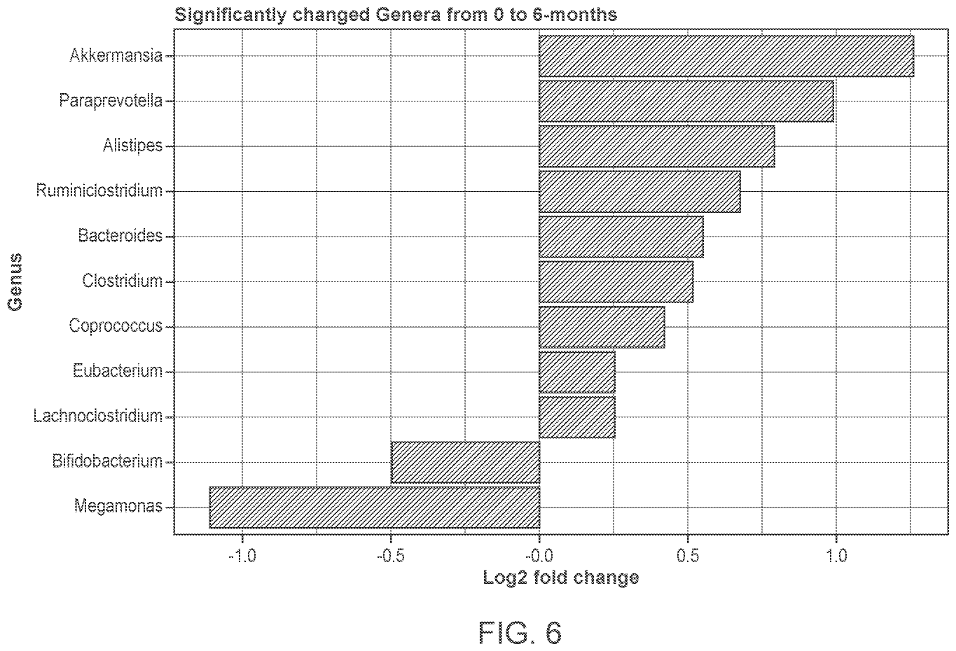

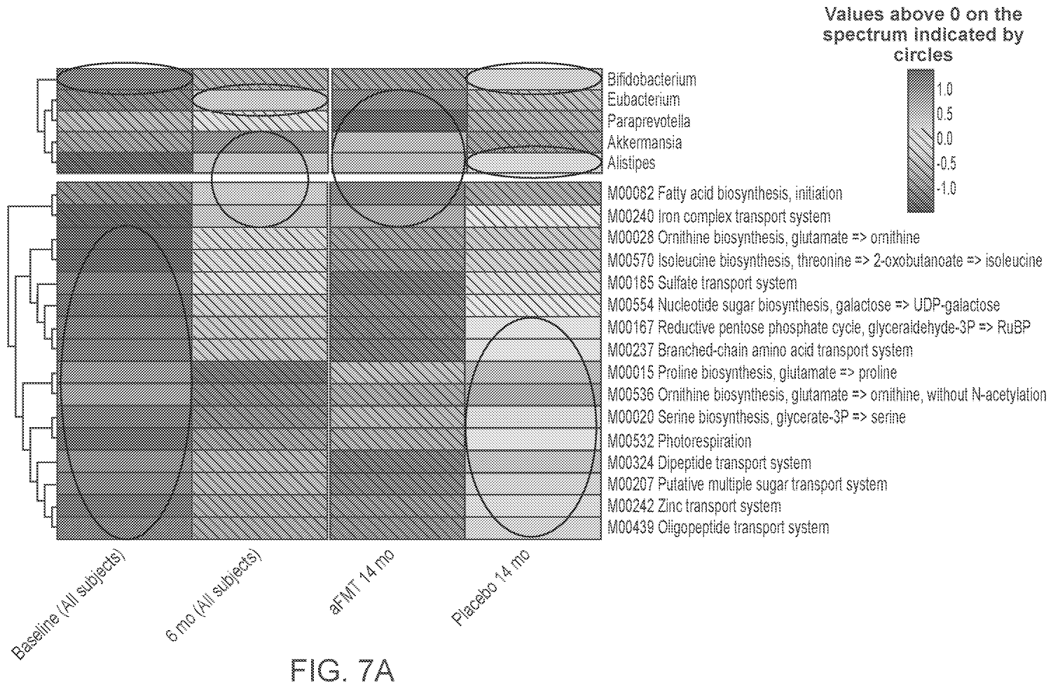

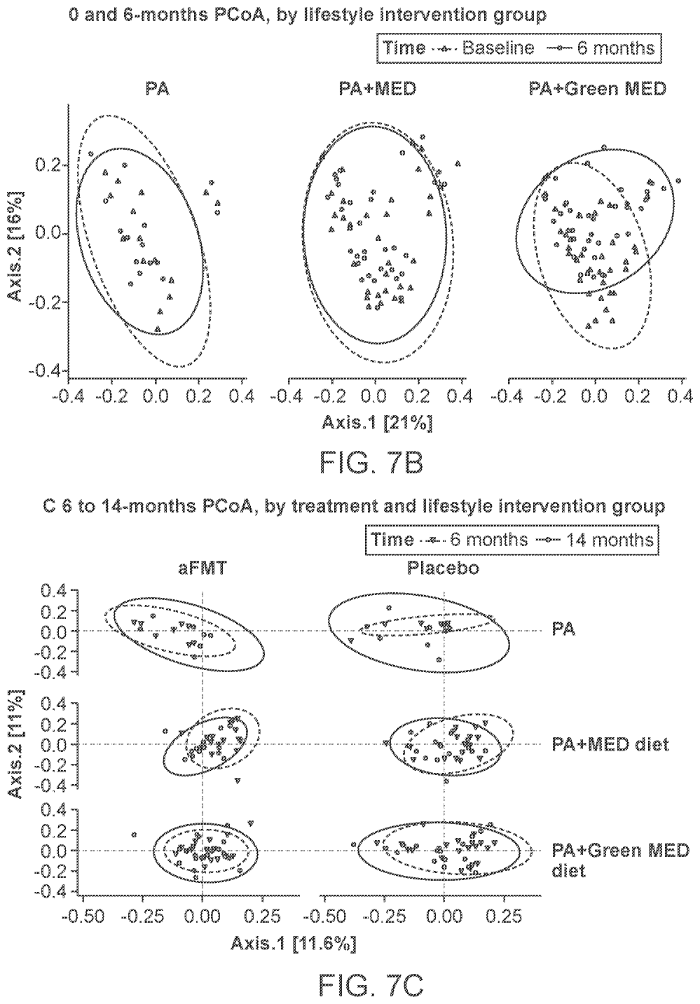

During the weight-loss phase, first 60 pathways and 15 genera were significantly altered (preliminary data, partial list) (FIG. 6). Following the regain phase, 16 modules and 5 genera remained significantly changed in the aFMT group alone (18 modules and 1 bacterium retained the weight-loss-induced change in both groups). Of note Bifidobacterium and several microbial amino-acid transport systems and biosynthesis pathways, including ornithine, isoleucine, proline and serine biosynthesis were downregulated during the weight-loss phase and remained lower only in the aFMT group, whereas Akkermansia and free fatty acid biosynthesis was upregulated by weight loss and maintained during the aFMT treatment (FIG. 7A).

Comparing microbiome compositional shift by the weight-loss phase (0-6 months), across lifestyle intervention groups, a significant change could be observed between 0 and 6-month fecal samples in the PA+Green MED groups (P=0.003), but not in the PA+MED (P=0.25) and PA (P=0.63) groups (FIG. 7B). The aFMT-Green-MED group had the most stable gut microbiome composition, though no significant differences were present between 6 and 14-month samples within any of the sub-groups (FIG. 7C).

aFMT significantly attenuated weight regain in the PA+Green-MED diet group [aFMT=+1.6 Kg (17.1%) vs. placebo=+3.5 kg (50.1%); P=0.03], but not in the PA and PA+MED groups (P=0.23 and 0.64, respectively; P=0.03 for the interaction between aFMT treatment and Green-MED diet). Similarly, waist circumference (WC) regain was significantly attenuated by aFMT within the Green-MED group (P=0.008; P of interaction=0.046), but not in the PA and PA+MED groups (P=0.32, P=0.33, respectively). We also observed a significant difference in fasting insulin levels between the aFMT and placebo groups in the PA+Green-MED group (P=0.04; P of interaction=0.03). A non-significant change in fasting insulin was noted between aFMT and placebo in the other intervention groups (Table 5).

TABLE 5
aFMT effect across lifestyle intervention; 6-14 months changes in body weight, waist
circumference and specific biomarkers

| | | | | | | | PA + Green MED | | | |
|---|---|---|---|---|---|---|---|---|---|---|
| | PA group | | | PA + MED group | | | group | | | P of |
| Treatment | aFMT | Placebo | P | aFMT | Placebo | P | aFMT | Placebo | P | interaction |
| Subjects-no. | 8 | 8 | | 17 | 18 | | 19 | 20 | | |
| ΔWeight-Kg | 1.94 | 1.43 | 0.297 | 3.09 | 2.77 | 0.779 | 1.57 | 3.45 | 0.029 | 0.13 |
| | (3.32) | (1.81) | | (3.97) | (4.03) | | (3.11) | (3.08) | | |
| Weight regain- | 43.32 | 34.51 | 0.355 | 39.35 | 33.02 | 0.621 | 17.13 | 50.11 | 0.022 | 0.033 |
| %** | (52.11) | (51.84) | | (39.85) | (41.43) | | (42.78) | (42.88) | | |
| ΔWaist | 1.62 | −0.43 | 0.323 | 2.71 | 2.06 | 0.336 | 2.39 | 4.72 | 0.018 | 0.043 |
| circumference- | (4.27) | (4.58) | | (4.33) | (5.92) | | (4.51) | (3.41) | | |
| cm | | | | | | | | | | |
| ΔFasting or | 1.56 | −0.66 | 0.208 | 0.88 | 0.80 | 0.468 | −1.46 | 1.64 | 0.044 | 0.032 |
| post prandial/ | (3.64) | (3.56) | | (3.18) | (4.92) | | (3.57) | (4.73) | | |
| load plasma | | | | | | | | | | |
| insulin- | | | | | | | | | | |
| microU/ml | | | | | | | | | | |
| ΔFasting or | 10.71 | 6.31 | 0.105 | 4.65 | 4.71 | 0.531 | 3.88 | 6.03 | 0.564 | 0.967 |
| post prandial/ | (10.22) | (1.90) | | (5.76) | (9.52) | | (10.95) | (9.94) | | |
| load plasma | | | | | | | | | | |
| glucose- mg/dl | | | | | | | | | | |

Values are presented as means (standard deviation).
*To detect overall P differences between treatment groups, we performed t-tests for normally distributed variables, ordinal variables and data determined to be non-normal after log-transformation were analyzed using Mann-Whitney test. A linear regression model with analysis of variance (ANOVA) was applied to assess interaction between the Green-MED diet and aFMT. A multivariate linear regression model was used to assess aFMT effect on metabolic parameters, adjusting for weight change.
*Regain % was calculated as 100 * (14-months-6-months)/(6-months-0-months).
#Significant difference was observed between placebo groups by Kruskal Wallis test
No other significant differences were observed within the placebo or aFMT group, across lifestyle interventions.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgement or any form of suggestion that they constitute valid prior art or form part of the common general knowledge in any country in the world.

### Embodiments

1. A method of altering/optimizing the gut microbiome of a first subject, the method comprising: administering to the first subject, a composition comprising one or more species of duckweed.

2. The method of embodiment 1, comprising: administering the duckweed/algae to the first subject until the first subject achieves an optimal/desired weight loss.

3. The method of embodiment 1 or 2, further comprising obtaining one or more microbiome sample from the gastrointestinal tract of the first subject once the optimal/desired weight loss is achieved.

4. The method of any one of embodiments 1-3, further comprising administering the one or more microbiome sample to the first subject after the optimal/desired weight loss is achieved, and over a period of weight maintenance.

5. The method of any one of embodiments 1-3, further comprising administering the one or more microbiome sample to a second subject.

6. The method of any one of embodiments 1-5, wherein a first population of microbes present in the gut microbiome before the administration of the composition increases in abundance/activity after the composition is administered; and/or wherein a second population of microbes present in the gut microbiome before the administration of the composition decreases in abundance/activity after the composition is administered.

7. The method of any one of embodiments 1-6, wherein a first population of microbes present in the gut microbiome before the administration of the composition increases in abundance/activity after the optimal weight loss is achieved; and/or wherein a second population of microbes present in the gut microbiome before the administration of the composition decreases in abundance after the optimal weight loss is achieved.

8. The method of any one of embodiments 1-7, wherein the gut microbiome of the subject comprises a higher proportion of bacteria belonging to at least one genus selected from the group consisting of Akkermansia, Paraprevotelia, Alistipes, Ruminiclostridium, Bacteroides, Clostridium, Coprococcus, Eubacterium and Lachnoclostridium, after the optimal weight loss is achieved, as compared to before the administration of the composition.

9. The method of any one of embodiments 1-8, wherein the gut microbiome of the subject comprises a lower proportion of bacteria belonging to at least one genus selected from the group consisting of Bifidobacterium and Megamonas, after the optimal weight loss is achieved, as compared to before the administration of the composition.

10. The method of any one of embodiments 1-9, wherein the gut microbiome of the subject comprises a higher proportion of bacteria belonging to at least one genus selected from the group consisting of Akkermansia, Paraprevotelia, Eubacterium and Alistipes, at the end of the period of weight maintenance, as compared to before the administration of the composition.

11. The method of any one of embodiments 1-10, wherein the gut microbiome of the subject comprises a lower proportion of bacteria belonging to at least one genus selected from the group consisting of Bifidobacterium, at the end of the period of weight maintenance, as compared to before the administration of the composition.

12. The method of any one of embodiments 1-11, wherein the one or more species of duckweed belongs to a genus selected from the group consisting of Landoltia, Lemna, Spirodela, Wolffia, and Wolffiella.

13. The method of any one of embodiments 1-12, wherein the one or more species of duckweed is Wolffia globosa.

14. The method of any one of embodiments 1-13, wherein the Wolffia globosa is the

Mankai varietal.

15. The method of any one of embodiments 1-14, wherein the one or more species of duckweed are fresh, dehydrated, blended, ground, chopped, frozen, or powdered.

16. The method of any one of embodiments 1-15, wherein the composition comprises one or more species of algae.

17. The method of any one of embodiments 1-16, wherein the composition is administered as a tablet, a pill, or a capsule.

18. The method of any one of embodiments 1-17, wherein the composition comprises an edible product such as a powder, a bar, a smoothie, a yogurt, a frozen/fresh/dry food preparation, or a shake.

19. The method of any one of embodiments 1-18, wherein one or more species of duckweed comprises at least one endophyte selected from the group consisting of Microbacterium arthrosphaerae, Bacillus niacin, Hymenobacter gelipurpurascens, Arthrobacter agilis, Moraxella_sg, Moraxella osloensis, Terribacillus aidingensis, Terribacillus saccharophilus, Virgibacillus picturae, and Pantoea septica.

20. A method of attenuating the rebound or regain of at least one weight loss-associated metabolic parameter after a period of weight loss in a first subject, the method comprising administering to the first subject a composition comprising one or more species of duckweed during the period of weight loss.

21. The method of embodiment 20, further comprising obtaining one or more microbiome sample from the gastrointestinal tract of the first subject at the end of the period of weight loss.

22. The method of embodiment 20 or 21, further comprising administering the one or more microbiome sample to the first subject after the end of the period of weight loss, and over a period of weight maintenance.

23. The method of embodiment 20 or 21, further comprising administering the one or more microbiome sample to a second subject.

24. The method of any one of embodiments 20-23, wherein the at least one weight loss-associated metabolic parameter is selected from the level of fasting or non-fasting or post prandial/load plasma insulin, the level of fasting or non-fasting or post prandial/load plasma glucose and waist circumference.

25. The method of any one of embodiments 20-24, wherein the at least one weight loss-associated metabolic parameter is the level of fasting or non-fasting or post prandial/load plasma insulin.

26. The method of any one of embodiments 20-25, wherein the rebound % of the level of fasting or non-fasting or post prandial/load plasma insulin in the subject is lower than the rebound % of the level of fasting or non-fasting or post prandial/load plasma insulin in a control subject, wherein the control subject is not administered the one or more species of duckweed over the period of weight loss.

27. The method of any one of embodiments 20-26, wherein the at least one weight loss-associated metabolic parameter is the level of fasting or non-fasting or post prandial/load plasma glucose.

28. The method of any one of embodiments 20-27, wherein the rebound % of the level of fasting or non-fasting or post prandial/load plasma glucose in the subject is lower than the rebound % of the level of fasting or non-fasting or post prandial/load plasma glucose in a control subject, wherein the control subject is not administered the one or more species of duckweed over the period of weight loss.

29. The method of any one of embodiments 20-28, wherein the at least one weight loss-associated metabolic parameter is waist circumference.

30. The method of any one of embodiments 20-29, wherein the regain % of the waist circumference of the subject is lower than the regain % of the waist circumference of a control subject, wherein the control subject is not administered the one or more species of duckweed over the period of weight loss.

31. The method of any one of embodiments 20-30, wherein the one or more species of duckweed belongs to a genus selected from the group consisting of Landoltia, Lemna, Spirodela, Wolffia, and Wolffiella.

32. The method of any one of embodiments 20-31, wherein the one or more species of duckweed is Wolffia globosa.

33. The method of any one of embodiments 20-32, wherein the Wolffia globosa is the Mankai varietal.

34. The method of any one of embodiments 20-33, wherein the one or more species of duckweed are fresh, dehydrated, blended, ground, chopped, frozen, or powdered.

35. The method of any one of embodiments 20-34, wherein the composition comprises one or more species of algae.

36. The method of any one of embodiments 20-35, wherein the composition is administered as a tablet, a pill, or a capsule.

37. The method of any one of embodiments 20-36, wherein the composition comprises an edible product such as a powder, a bar, a smoothie, a yogurt, a frozen/fresh/dry food preparation, or a shake.

38. The method of any one of embodiments 20-37, wherein one or more species of duckweed comprises at least one endophyte selected from the group consisting of Microbacterium arthrosphaerae, Bacillus niacin, Hymenobacter gelipurpurascens, Arthrobacter agilis, Moraxella_sg, Moraxella osloensis, Terribacillus aidingensis, Terribacillus saccharophilus, Virgibacillus picturae, and Pantoea septica.

39. A method for treating obesity or an obesity-related disorder or a weight-related disorder in a first subject, the method comprising administering to the first subject a composition comprising one or more species of duckweed.

40. The method of embodiment 39, comprising: administering the duckweed to the first subject until the subject achieves an optimal weight loss.

41. The method of embodiment 39 or 40, further comprising: obtaining one or more microbiome sample from the gastrointestinal tract of the first subject once the optimal weight loss is achieved.

42. The method of any one of embodiments 39-41, further comprising administering the one or more microbiome sample to the first subject after the optimal weight loss is achieved, and over a period of weight maintenance.

43. The method of any one of embodiments 39-41, further comprising administering the one or more microbiome sample to a second subject.

44. The method of any one of embodiments 39-43, wherein the obesity-related disorder is diabetes, prediabetes, dyslipidemia, heart disease, stroke, high blood pressure, cancer, gallbladder disease, osteoarthritis, gout, sleep apnea, fatty liver diseases, kidney disease, a breathing problem, such as asthma; a pregnancy problem such as gestational diabetes, preeclampsia, and requiring Cesarean delivery (C-section), requiring longer recovery after birth; mental illness such as clinical depression, anxiety, and other mental disorders; difficulty with physical functioning; body pain, or any combination thereof.

45. The method of any one of embodiments 39-44, wherein the one or more species of duckweed belongs to a genus selected from the group consisting of Landoltia, Lemna, Spirodela, Wolffia, and Wolffiella.

46. The method of any one of embodiments 39-45, wherein the one or more species of duckweed is Wolffia globosa.

47. The method of any one of embodiments 39-46, wherein the Wolffia globosa is the Mankai varietal.

48. The method of any one of embodiments 39-47, wherein the one or more species of duckweed are fresh, dehydrated, blended, ground, chopped, frozen, or powdered.

49. The method of any one of embodiments 39-48, wherein the composition comprises one or more species of algae.

50. The method of any one of embodiments 39-49, wherein the composition is administered as a tablet, a pill, or a capsule.

51. The method of any one of embodiments 39-50, wherein the composition comprises an edible product such as a powder, a bar, a smoothie, a yogurt, a frozen/fresh/dry food preparation, or a shake.

52. The method of any one of embodiments 39-51, wherein one or more species of duckweed comprises at least one endophyte selected from the group consisting of Microbacterium arthrosphaerae, Bacillus niacin, Hymenobacter gelipurpurascens, Arthrobacter agilis, Moraxella_sg, Moraxella osloensis, Terribacillus aidingensis, Terribacillus saccharophilus, Virgibacillus picturae, and Pantoea septica.

53. A method for treating an age-related disorder or deterioration in a subject, the method comprising administering to the first subject a composition comprising one or more species of duckweed.

54. The method of embodiment 53, wherein the age-related disorder or deterioration affects the physical health, cognition and/or fertility of the subject.

55. The method of embodiment 53 or 54, wherein the age-related disorder or deterioration is atherosclerosis, cardiovascular disease, cancer, arthritis, cataracts, osteoporosis, type 2 diabetes, hypertension, Alzheimer's disease, Parkinson's disease, dementia, Chronic Obstructive Pulmonary Disease (COPD), hearing loss, or any combination thereof.

56. A composition as described herein.

57. A method as described herein.

58. A method as described herein, which comprises administering a composition as described herein, wherein said result from the method can be found in any of the aforementioned examples and/or figures, said result being any number, metric, parameter, biomarker, etc. as specified herein.

1. A method of altering the gut microbiome of a subject, the method comprising: administering to the subject, a composition comprising one or more species of duckweed, wherein said one or more species are frozen or dry, wherein said composition is administered in an amount which achieves weight loss in the subject, thereby altering the gut microbiome of the subject.

2. (canceled)

3. The method of claim 1, further comprising:
(a) obtaining one or more microbiome sample from the gastrointestinal tract of the subject once the optimal/desired weight loss is achieved; and
(b) administering the one or more microbiome sample to the first after the optimal/desired weight loss is achieved, and over a period of weight maintenance.

4-11. (canceled)

12. The method of claim 1, wherein the one or more species of duckweed belongs to a genus selected from the group consisting of Landoltia, Lemna, Spirodela, Wolffia, and Wolffiella.

13. The method of claim 12, wherein the one or more species of duckweed is Wolffia globosa.

14. The method of claim 13, wherein the Wolffia globosa is of the Mankai variety.

15. (canceled)

16. The method of claim 1, wherein the composition comprises one or more species of algae.

17. The method of claim 1, wherein the composition is administered as a tablet, a pill, or a capsule.

18. The method of claim 1, wherein the composition is comprised in an edible product selected from the group consisting of a powder, a bar, a smoothie, a yogurt and a shake.

19. The method of claim 1, wherein one or more species of duckweed comprises at least one endophyte selected from the group consisting of Microbacterium arthrosphaerae, Bacillus niacin, Hymenobacter gelipurpurascens, Arthrobacter agilis, Moraxella_sg, Moraxella osloensis, Terribacillus aidingensis, Terribacillus saccharophilus, Virgibacillus picturae, and Pantoea septica.

20. A method of attenuating the regain of at least one weight loss-associated metabolic parameter after a period of weight loss in a subject, the method comprising administering to the subject a composition comprising one or more species of duckweed during the period of weight loss, thereby attenuating the regain of the at least one weight loss-associated metabolic parameter, wherein the duckweed is frozen or dry.

21. The method of claim 20, further comprising:
(a) obtaining one or more microbiome sample from the gastrointestinal tract of the subject at the end of the period of weight loss; and
(b) administering the one or more microbiome sample to the subject after the end of the period of weight loss, and over a period of weight maintenance.

22-23. (canceled)

24. The method of claim 20, wherein the at least one weight loss-associated metabolic parameter is selected from the level of fasting or non-fasting or post prandial/load plasma insulin, the level of fasting or non-fasting or post prandial/load plasma glucose and waist circumference.

25-30. (canceled)

31. The method of claim 20, wherein the one or more species of duckweed belongs to a genus selected from the group consisting of Landoltia, Lemna, Spirodela, Wolffia, and Wolffiella.

32. The method of claim 31, wherein the one or more species of duckweed is Wolffia globosa.

33. The method of claim 32, wherein the Wolffia globosa is of the Mankai variety.

34-37. (canceled)

38. The method of claim 20, wherein one or more species of duckweed comprises at least one endophyte selected from the group consisting of Microbacterium arthrosphaerae, Bacillus niacin, Hymenobacter gelipurpurascens, Arthrobacter agilis, Moraxella_sg, Moraxella osloensis, Terribacillus aidingensis, Terribacillus saccharophilus, Virgibacillus picturae, and Pantoea septica.

39. A method for treating obesity or an obesity-related disorder or a weight-related disorder in a first subject, the method comprising administering to the first subject a therapeutically effective amount of a composition comprising one or more species of duckweed, thereby treating the obesity.

40. The method of claim 39, comprising:
administering the duckweed to the subject until the subject achieves an optimal weight loss.

41. The method of claim 40, further comprising:
(a) obtaining one or more microbiome sample from the gastrointestinal tract of the first subject once the optimal weight loss is achieved; and
(b) administering the one or more microbiome sample to the subject after the optimal weight loss is achieved, and over a period of weight maintenance.

42-43. (canceled)

44. The method of claim 39, wherein the obesity-related disorder is diabetes, prediabetes, dyslipidemia, heart disease, stroke, high blood pressure, cancer, gallbladder disease, osteoarthritis, gout, sleep apnea, fatty liver diseases, kidney disease, a breathing problem, such as asthma; a pregnancy problem such as gestational diabetes, preeclampsia, and requiring Cesarean delivery (C-section), requiring longer recovery after birth; mental illness such as clinical depression, anxiety, and other mental disorders; difficulty with physical functioning; body pain, or any combination thereof.

45. The method of claim 39, wherein the one or more species of duckweed belongs to a genus selected from the group consisting of Landoltia, Lemna, Spirodela, Wolffia, and Wolffiella.

46. The method of claim 45, wherein the one or more species of duckweed is Wolffia globosa.

47. The method of claim 46, wherein the Wolffia globosa is of the Mankai variety.

48. The method of claim 39, wherein the one or more species of duckweed are frozen fresh, dehydrated, blended, ground, chopped, frozen, or powdered or dry.

49-52. (canceled)

53. A method for treating an age-related disorder or deterioration in a subject, the method comprising administering to the first subject a therapeutically effective amount of a composition comprising one or more species of duckweed, thereby treating the age-related disorder or deterioration.

54. The method of claim 53, wherein the age-related disorder or deterioration affects the physical health, cognition and/or fertility of the subject.

55. The method of claim 53, wherein the age-related disorder or deterioration is atherosclerosis, cardiovascular disease, cancer, arthritis, cataracts, osteoporosis, type 2 diabetes, hypertension, Alzheimer's disease, Parkinson's disease, dementia, Chronic Obstructive Pulmonary Disease (COPD), hearing loss, or any combination thereof.

56. The method of claim 53, wherein said one or more species of duckweed is frozen or dry.
